(19) United States
(12) Patent Application Publication
(10) Pub. No.: US 2018/0235505 A1
(43) Pub. Date: Aug. 23, 2018

(54) METHOD AND SYSTEM FOR INFERENCE OF EEG SPECTRUM IN BRAIN BY NON-CONTACT MEASUREMENT OF PUPILLARY VARIATION

(71) Applicants: SANGMYUNG UNIVERSITY INDUSTRY-ACADEMY COOPERATION FOUNDATION, Seoul (KR); CENTER OF HUMAN-CENTERED INTERACTION FOR COEXISTENCE, Seoul (KR)

(72) Inventors: Min Cheol WHANG, Goyang-si (KR); Sang In PARK, Seoul (KR); Myoung Ju WON, Cheonan-si (KR); Dong Won LEE, Seongnam-si (KR)

(73) Assignees: SANGMYUNG UNIVERSITY INDUSTRY-ACADEMY COOPERATION FOUNDATION, Seoul (KR); CENTER OF HUMAN-CENTERED INTERACTION FOR COEXISTENCE, Seoul (KR)

(21) Appl. No.: 15/869,626

(22) Filed: Jan. 12, 2018

(30) Foreign Application Priority Data
Feb. 17, 2017 (KR) 10-2017-0021519
Nov. 7, 2017 (KR) 10-2017-0147607

(51) Int. Cl. A61B 5/0484; A61B 5/04; A61B 5/0456

(52) U.S. Cl. CPC A61B 5/04845; A61B 5/4064; A61B 5/0456; A61B 5/04012

(57) ABSTRACT

Provided are a method and system for non-contact measurement of an electroencephalogram (EEG) spectrum based on pupillary variation. To infer the EEG spectrum from moving images of a subject's pupil, the method includes obtaining moving images of the pupil from the subject, extracting data of pupillary variation from the moving images, extracting a plurality of signals for a plurality of frequency bands based on frequency analysis, and calculating outputs of the plurality of the signals to be used as parameters of a brain-frequency domain.

### CROSS-REFERENCE TO RELATED APPLICATION

This application claims the benefit of Korean Patent Application Nos. 10-2017-0021519, filed on Feb. 7, 2017, and 10-2017-0147607, filed on Nov. 7, 2017, in the Korean Intellectual Property Office, the disclosures of which are incorporated herein in their entirety by reference.

### BACKGROUND

### 1. Field

Additional aspects will be set forth in part in the description which follows and, in part, will One or more embodiments relate to a method of inferring human physiological signals performed in a non-contact mode, and a system using the method, and more particularly, to method of detecting parameters of a brain-frequency domain from pupil rhythm video captured by a camera.

### 2. Description of the Related Art

In vital signal monitoring (VSM), physiological information can be acquired by a sensor attached to a human body. Such physiological information includes electrocardiogram (ECG), photo-plethysmograph (PPG), blood pressure (BP), galvanic skin response (GSR), skin temperature (SKT), respiration (RSP) and electroencephalogram (EEG).

The heart and brain are two main organs of the human body and analysis thereof provide the ability to evaluate human behavior and obtain information that may be used in response to events and in medical diagnosis. The VSM may be applicable in various fields such as ubiquitous healthcare (U-healthcare), emotional information and communication technology (e-ICT), human factor and ergonomics (HF&E), human computer interfaces (HCIs), and security systems.

ECG and EEG use sensors attached to the body to measure physiological signals and thus, may cause inconvenience to patients. That is, the human body experiences considerable stress and inconvenience when using sensors to measure the signals. In addition, there are burdens and restrictions with respect to the cost of using the attached sensor and the movement of the subject due to attached hardware such as the sensors.

Therefore, VSM technology is required in the measurement of physiological signals by using non-contact, non-invasive, and non-obtrusive methods while providing unfettered movement at low cost.

Recently, VSM technology has been incorporated into wireless wearable devices allowing for the development of portable measuring equipment. These portable devices can measure the heart rate (HR) and RSP by using VSM embedded into accessories such as watches, bracelets, or glasses.

Wearable device technology is predicted to transit from portable devices to “attachable” devices shortly. It is predicted that attachable devices will transit to “eatable” devices.

VSM technology has been developed to measure physiological signals by using non-contact, non-invasive, and non-obtrusive methods that provides unfettered movement at low cost. While VSM will continue to advance technologically, innovative vision-based VSM technology is required to be developed also.

### SUMMARY

One or more embodiments include a system and method for inferring and detecting physiological signals by non-contact, non-invasive and non-obstructive method at low cost.

In detail, one or more embodiments include a system and method for detecting parameters of a brain frequency domain by using rhythm of pupillary variation.

Additional aspects will be set forth in part in the description which follows and, in part, will be apparent from the description, or may be learned by practice of the presented embodiments.

According to one or more exemplary embodiments, the method of inferring EEG spectrum based on pupillary variation comprises: obtaining moving images of at least one pupil from a subject; extracting data of pupillary variation from the moving images; extracting band data for a plurality of frequency bands to be used as brain frequency information, based on frequency analysis of the signal of pupillary variation; and calculating outputs of the band data to be used as parameters of a brain-frequency domain.

According to one or more exemplary embodiment, the data of pupillary variation comprises a signal indicating pupil size variation of the subject.

According to one or more exemplary embodiment, the frequency analysis is performed in a range of 0.01 Hz-0.50 Hz.

According to one or more exemplary embodiment, the method further comprises resampling of the data of pupillary variation at a predetermined sampling frequency, before extracting the band data based on the frequency analysis.

According to one or more exemplary embodiment, the plurality of frequency bands include at least one of: a delta range of 0.01 Hz˜0.04 Hz, a theta range of 0.04 Hz˜0.08 Hz, an alpha range of 0.08 Hz˜0.13 Hz, a beta range of 0.13 Hz˜0.30 Hz, a gamma range of 0.30 Hz˜0.50 Hz, a slow alpha range of 0.08 Hz˜0.11 Hz, a fast alpha range of 0.11 Hz˜0.13 Hz, a low beta range of 0.12 Hz˜0.15 Hz, a mid beta range of 0.15 Hz˜0.20 Hz, a high beta range of 0.20 Hz˜0.30 Hz, a mu range of 0.09 Hz˜0.11 Hz, a SensoriMotor Rhythm (SMR) wave range of 0.125 Hz˜0.155 Hz, and a total band range of 0.01 Hz˜0.50 Hz.

According to one or more exemplary embodiment, each of the outputs is obtained from a ratio of respective band power to total band power of a total band range in which the plurality of frequency bands are included.

According to one or more exemplary embodiment, the system adopting the method, comprises: video equipment configured to capture the moving images of the subject; and a computer architecture based analyzing system, including analysis tools, configured to process and analyze the moving images in the plurality of frequency bands.

According to one or more exemplary embodiment, the analyzing system is configured to perform frequency analysis in a range of 0.01 Hz-0.50 Hz.

According to one or more exemplary embodiment, the range includes at least one of: a delta range of 0.01 Hz˜0.04 Hz, a theta range of 0.04 Hz˜0.08 Hz, an alpha range of 0.08 Hz˜0.13 Hz, a beta range of 0.13 Hz˜0.30 Hz, a gamma range of 0.30 Hz˜0.50 Hz, a slow alpha range of 0.08 Hz˜0.11 Hz, a fast alpha range of 0.11 Hz˜0.13 Hz, a low beta range of 0.12 Hz˜0.15 Hz, a mid beta range of 0.15 Hz˜0.20 Hz, a high beta range of 0.20 Hz˜0.30 Hz, a mu range of 0.09 Hz˜0.11 Hz, a SMR wave range of 0.125 Hz˜0.155 Hz, and a total band range of 0.01 Hz˜0.50 Hz.

### BRIEF DESCRIPTION OF THE DRAWINGS

In These and/or other aspects will become apparent and more readily appreciated from the following description of the embodiments, taken in conjunction with the accompanying drawings in which:

FIG. 1 shows a procedure for selecting a representative of sound stimulus used in an exemplary test, according to one or more embodiments;

FIG. 2 shows an experimental procedure for measuring the amount of movement in an upper body, according to one or more embodiments;

FIG. 3 is a block diagram for explaining an experiment procedure, according to one or embodiments;

FIG. 4 shows a procedure for detecting the pupil region, according to one or more embodiments;

FIG. 5A shows a procedure of signal processing an electroencephalogram (EEG) spectral index from pupillary response, according to one or more embodiments;

FIG. 5B shows a procedure of signal processing an EEG spectral index from an EEG signal, according to one or more embodiments;

FIG. 6 shows a result of statistical analysis of an average amount of movement in an upper body in a movelessness condition (MNC) and natural movement condition (NMC), according to one or more embodiments;

FIGS. 7A and 7B show an experiment procedure for detecting the spectral index from pupillary response and EEG signals (ground truth) respectively, according to one or more embodiments;

FIG. 8 shows comparisons of the EEG spectral indices (frontal cortex) of the pupillary response and EEG signal in a state of MNC, according to one or more embodiments;

FIG. 9 shows comparisons of the EEG spectral indices (parietal and central cortex) of the pupillary response and EEG signal in a state of MNC, according to one or more embodiments;

FIG. 10 shows comparisons of the EEG spectral indices (frontal cortex) of the pupillary response and EEG signal in a state of NMC, according to one or more embodiments;

FIG. 11 shows comparisons of the EEG spectral indices (parietal and central cortex) of the pupillary response and EEG signal in a state of NMC, according to one or more embodiments;

FIG. 12 shows an example of an infrared web-cam system for measuring a pupil image, according to one or more embodiments; and

FIG. 13 shows an example of a graphic user interface of an infrared web-cam system for measuring pupil images, according to one or more embodiments.

### DETAILED DESCRIPTION

In Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Hereinafter, a method and system for inferring and detecting physiological signals according to the present inventive concept is described with reference to the accompanying drawings.

The invention may, however, be embodied in many different forms and should not be construed as being limited to the embodiments set forth herein; rather, these embodiments are provided so that this disclosure will be thorough and complete, and will fully convey the concept of the invention to those skilled in the art. Like reference numerals in the drawings denote like elements. In the drawings, elements and regions are schematically illustrated. Accordingly, the concept of the invention is not limited by the relative sizes or distances shown in the attached drawings.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, numbers, steps, operations, elements, and/or components, but do not preclude the presence or addition of one or more other features, numbers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present application, and will not be interpreted in an overly formal sense unless expressly so defined herein.

The embodiments described below involve processing brain frequency information from pupillary response which is obtained from video information

The present invention, which may be sufficiently understood through the embodiments described below, involve extraction brain frequency information from the pupillary response by using a vision system equipped with a video camera such as a webcam without any physical restriction or psychological pressure on the subject, Especially, the pupil response is detected from the image information and brain frequency information is extracted from it.

In the experiment of the present invention, the reliability of the parameters of the brain frequency domain extracted from the pupil size variation (PSV) acquired through moving images was compared with the EEG signal by EEG of ground truth.

The experiment of the present invention has been performed by video equipment, and computer architecture based analyzing system for processing and analyzing the moving image which includes analysis tools provided by software.

### Experimental Stimuli

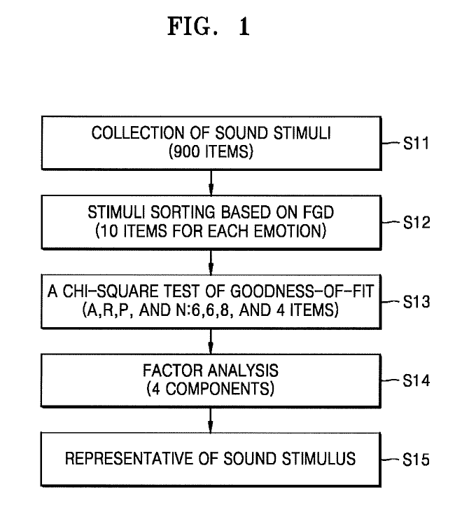

In order to cause variation of the physiological state, this experiment used sound stimuli based on the Russell's cir-complex model (Russell, 1980). The sound stimuli included a plurality of factors, including arousal, relaxation, positive, negative, and neutral sounds. The neutral sound was defined by an absence of acoustic stimulus. The steps for selecting sound stimulus are shown in FIG. 1 and listed as follows:

(S11) Nine hundred sound sources were collected from the broadcast media such as advertisements, dramas, and movies.

(S12) The sound sources were then categorized into four groups (i.e., arousal, relaxation, positive, and negative). Each group was comprised of 10 commonly selected items based on a focus group discussion for a total of forty sound stimuli.

(S13) These stimuli were used to conduct surveys for suitability for each emotion (i.e., A: arousal, R: relaxation, P: positive, and N: negative) based on data gathered from 150 subjects that were evenly split into 75 males and 75 females. The mean age was 27.36 years±1.66 years. A subjective evaluation was required to select each item for the four factors, which could result in duplicates of one or more of the items.

(S14) A chi-square test for goodness-of-fit was performed to determine whether each emotion sound was equally preferred. Preference for each emotion sound was equally distributed in the population (arousal: 6 items, relaxation: 6 items, positive: 8 items, and negative: 4 items) as shown in Table 1.

Table 1 shows the chi-square test results for goodness-of-fit in which the items selected for each emotion are based on comparisons of observation and expectation values.

TABLE 1

| | N | Chi-Square | Sig. |
|---|---|---|---|
| Arousal | | | |
| arousal 1 | 150 | 83.867 | .000 |
| arousal 2 | 150 | 45.573 | .000 |
| arousal 3 | 150 | 58.200 | .000 |
| arousal 5 | 150 | 83.440 | .000 |
| arousal 9 | 150 | 10.467 | .000 |
| arousal 10 | 150 | 70.427 | .000 |
| Relaxation | | | |
| relaxation 1 | 150 | 131.120 | .000 |
| relaxation 2 | 150 | 163.227 | .000 |
| relaxation 5 | 150 | 80.720 | .000 |
| relaxation 6 | 150 | 11.640 | .000 |
| relaxation 7 | 150 | 82.587 | .000 |
| relaxation 10 | 150 | 228.933 | .000 |
| Positive | | | |
| positive 2 | 150 | 35.040 | .000 |
| positive 3 | 150 | 90.533 | .000 |
| positive 4 | 150 | 101.920 | .000 |
| positive 5 | 150 | 66.040 | .000 |
| positive 7 | 150 | 143.813 | .000 |
| positive 8 | 150 | 128.027 | .000 |
| positive 9 | 150 | 47.013 | .000 |
| positive 10 | 150 | 138.053 | .000 |
| Negative | | | |
| negative 1 | 150 | 119.920 | .000 |
| negative 2 | 150 | 59.440 | .000 |
| negative 5 | 150 | 117.360 | .000 |
| negative 9 | 150 | 62.080 | .000 |

Resurveys of the sound stimuli were conducted for relation to each emotion from the 150 subjects by using a seven-point scale based on 1 indicating strong disagreement to 7 indicating strong agreement.

Valid sounds relating to each emotion were analyzed using PCA (Principal Component Analysis) based on Varimax (orthogonal) rotation. The analysis yielded four factors explaining of the variance for the entire set of variables. Following the analysis result, representative sound stimuli for each emotion were derived, as shown in Table 2.

In Table 2, the bold type is the same factor, the blur character is the communalities <0.5, and the thick, light gray lettering with shading in the background represents the representative acoustic stimulus for each emotion.

### Experimental Procedure

In Seventy undergraduate volunteers of both genders, evenly split between males and females, ranging in age from 20 to 30 years old with a mean of 24.52 years±0.64 years participated in this experiment. All subjects had normal or corrected-to-normal vision (i.e., over 0.8), and no family or medical history of disease involving visual function, cardiovascular system, or the central nervous system. Informed written consent was obtained from each subject prior to the study. This experimental study was approved by the Institutional Review Board of Sangmyung University, Seoul, South Korea (2015 Aug. 1).

The experiment was composed of two trials where each trail was conducted for a duration of 5 min. The first trail was based on the movelessness condition (MNC), which involves not moving or speaking. The second trial was based on the natural movement condition (NMC) involving simple conversations and slight movements. Participants repeatedly conducted the two trials and the order was randomized across the subjects. In order to verify the difference of movement between the two conditions, this experiment quantitatively measured the amount of movement during the experiment by using webcam images of each subject. In the present invention, the moving image may include at least one pupil, that is, one pupil or both pupils image.

The images were recorded at 30 frames per second (fps) with a resolution of 1920×1080 by using a HD Pro C920 camera from Logitech Inc. The movement measured the upper body and face based on MPEG-4 (Tekalp and Ostermann, 2000; JPandzic and Forchheimer, 2002). The movement in the upper body was extracted from the whole image based on frame differences. The upper body line was not tracking because the background was stationary.

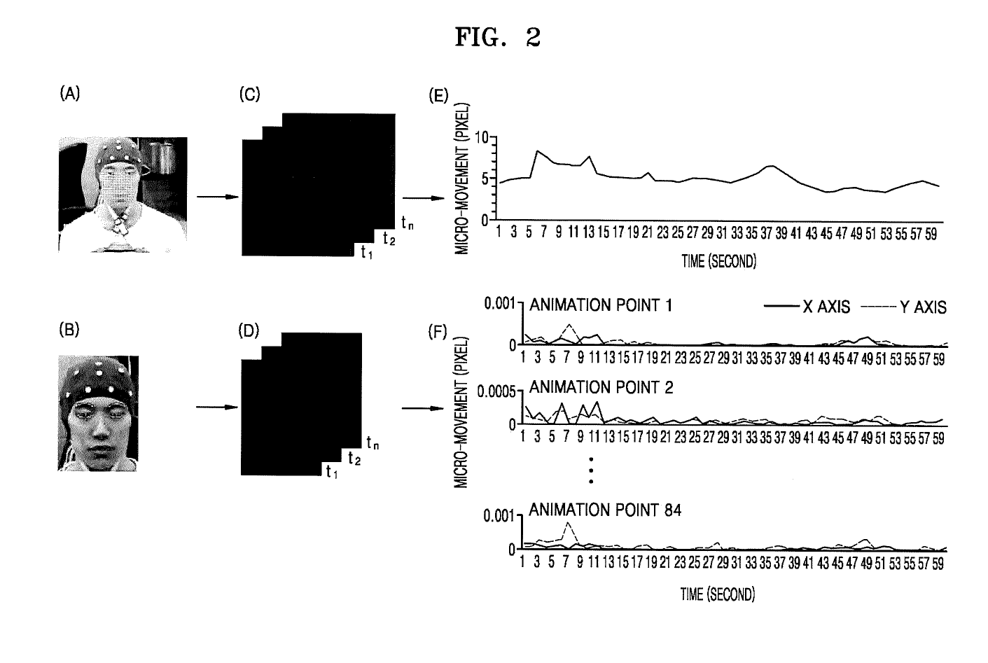

The movement in the face was extracted from 84 MPEG-4 animation points based on frame differences by using visage SDK 7.4 software from Visage Technologies Inc. All movement data used the mean value from each subject during the experiment and was compared to the difference of movement between the two trails, as shown in FIG. 2.

FIG. 2 shows an example of measuring the amount of motion of the subject's upper body in a state of the face is located at the intersection of the X axis and the Y axis,

In FIG. 2, (A) is an upper body image, (B) is a tracked face image at 84 MPEG-4 animation points, (C) and (D) shows the difference between before and after frames, (E) is a movement signal from the upper body, and (F) shows movement signals from 84 MPEG-4 animation points.

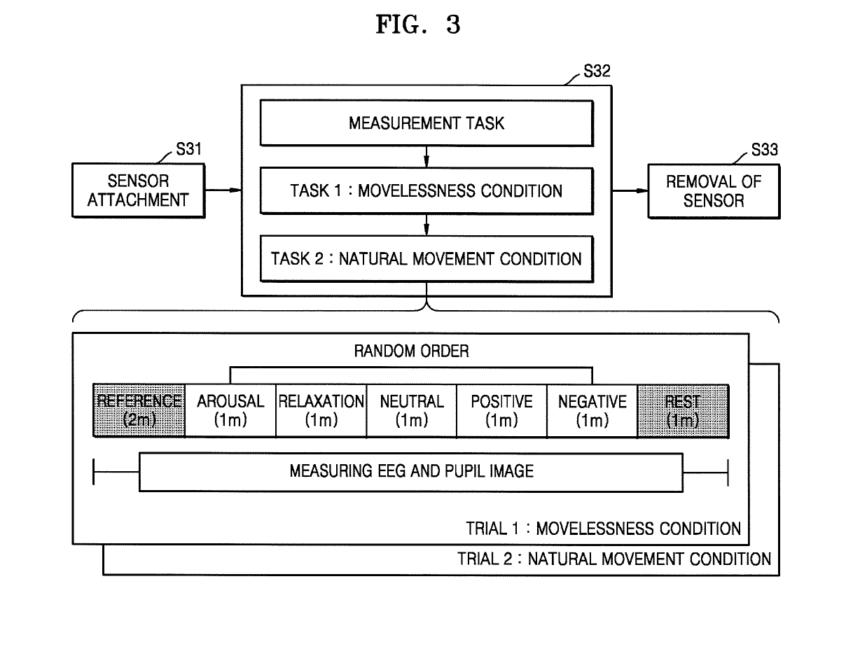

In order to cause the variation of physiological states, sound stimuli were presented to the participants during the trails. Each sound stimulus was randomly presented for 1 min for a total of five stimuli over the 5 min trial. A reference stimulus was presented for 3 min prior to the initiation of the task. The detailed experimental procedure is shown in FIG. 3.

The experimental procedure includes the sensor attachment S31, the measurement task S32 and the sensor removal S33 as shown in FIG. 3, and the measurement task S32 proceed as follows.

The experiment was conducted indoors with varying illumination caused by sunlight entering through the windows. The participants gazed at a black wall at a distance of 1.5 m while sitting in a comfortable chair. Sound stimuli were equally presented in both the trials by using earphones. The subjects were asked to constrict their movements and speaking during the movelessness trial (MNC). However, the natural movement trial (NMC) involved a simple conversation and slight movement by the subjects. The subjects were asked to introduce themselves to another person as part of the conversation for sound stimuli thereby involving feelings and thinking of the sound stimuli. During the experiment, EEG signal and pupil image data were obtained. EEG signals were recorded at a 500 Hz sampling rate from nineteen channels (FP1, FP2, F3, Fz, F4, F7, F8, C3, Cz, C4, T7 (T3), T8 (T4), P7 (T5), P8 (T6), P3, Pz, P4, O1, and O2 regions) based on the international 10-20 system (ground: FAz, reference: average between electrodes on the two ears, and DC level: 0 Hz-150 Hz). The electrode impedance was kept below 3 kΩ. EEG signals were recorded at 500 Hz sampling rate using a Mitsar-EEG 202 Machine.

Hereinafter, a method of extracting or constructing (recovering) a vital sign from a pupillary response will be described.

### Extraction of Pupillary Response

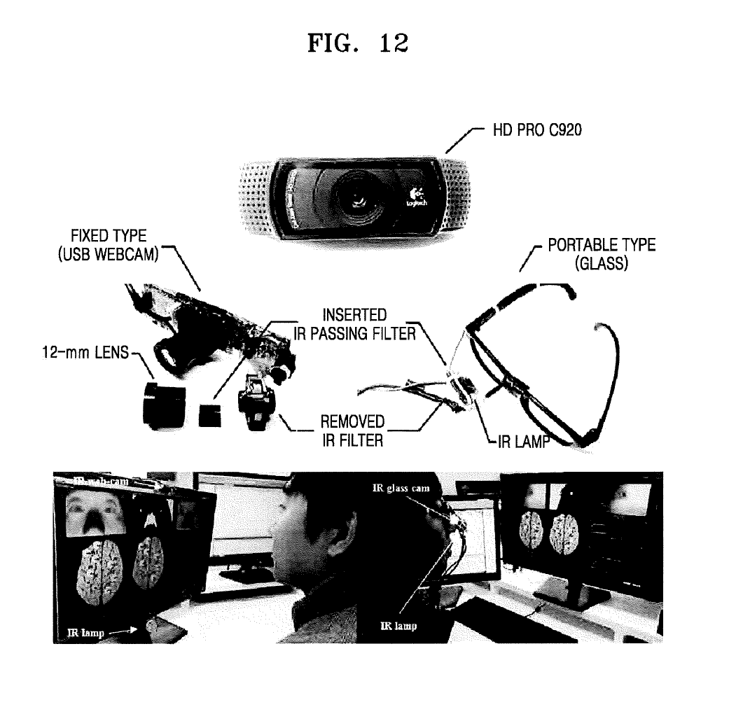

The pupil detection procedure acquires a moving image using the infrared camera system as shown in FIG. 12, and then requires a specific image processing procedure

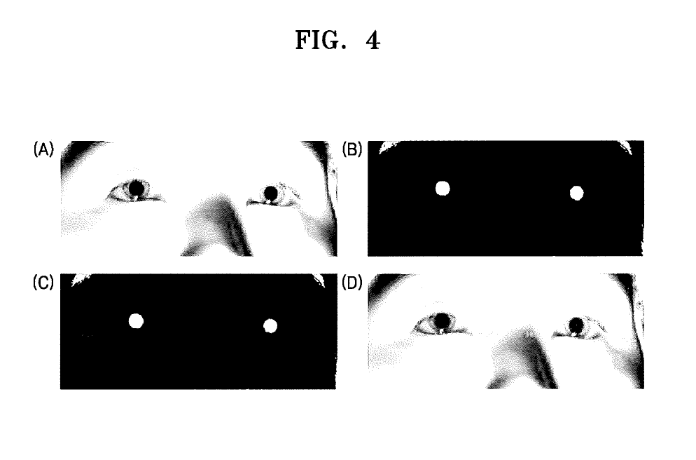

The pupil detection procedure required following certain image processing steps since the images were captured using an infrared camera, as shown in FIG. 4.

FIG. 4 shows a process of detecting a pupil region from the face image of a subject. In FIG. 4, (A) show an input image (gray scale) obtained from a subject, (B) show a binarized image based on an auto threshold, (C) shows a pupil position by circular edge detection, and (D) shows the real-time detection result of the pupil region including the information about the center coordinates and the diameter of the pupil region. The threshold value was defined by a linear regression model that used a brightness value of the whole image, as shown in Equation 1.

Threshold=(−0.418×Bₘₑₐₙ+1.051×Bₘₐₓ)+7.973 <Equation 1>

B=Brightness value

The next step to determine the pupil position involved processing the binary image by using a circular edge detection algorithm, as shown in Equation 2 (Daugman, 2004; Lee et al., 2009).

$${Max}_{( r , {x}_{0} , {y}_{0} )} \vert G σ ( r ) * \frac{δ}{δ r} {∮}_{r , {x}_{0} , {y}_{0}} \frac{I ( x , y )}{2 π r} ds \vert \\ I ( x , y ) = a grey level at the ( x , y ) position \\ ( {x}_{0} , {y}_{0} ) = center postion of pupil \\ r = radius of pupil \qquad < Equation 2 >$$

In case that multiple pupil positions were selected, the reflected light caused by the infrared lamp was used. Then we obtained an accurate pupil position, including centroid coordinates (x, y) and a diameter.

Pupil diameter data (signal) was resampled at a frequency range of 1 Hz-30 Hz, as shown in Equation 3. The resampling procedure for the pupil diameter data involved a sampling rate of 30 data points, which then calculated the mean value during 1-s intervals by using a common sliding moving average technique (i.e., a window size of 1 second and a resolution of 1 second). However, non-tracked pupil diameter data caused by the eye closing was not involved in the resampling procedure.

$${( {SMA}_{m} )}_{x + n} = {( \frac{{\sum }_{i = 1}^{m} {P}_{i}}{m} )}_{x} , {( \frac{{\sum }_{i = 1}^{m} {P}_{i}}{m} )}_{x + 1} , … , \\ {( \frac{{\sum }_{i = 1}^{m} {P}_{i}}{m} )}_{x + n} \\ SMA = sliding moving average \\ P = pupil diameter \qquad < Equation 3 >$$

### Detecting the EEG Spectral Index in Brain Activity

The non-contact detecting or inferring of the EEG spectral index is proposed in this section.

The index includes delta (δ, 1 Hz-4 Hz), theta (θ, 4 Hz-8 Hz), alpha (α, 8 Hz-13 Hz); beta (β, 13 Hz-30 Hz), gamma (γ, 30 Hz-50 Hz), slow alpha (8 Hz-11 Hz), fast alpha (11 Hz-13 Hz), low beta (12 Hz-15 Hz), mid beta (15 Hz-20 Hz), high beta (20 Hz-30 Hz), mu (μ, 9 Hz-11 Hz), and the sensorimotor rhythm wave (SMR) (12.5 Hz-15.5 Hz) by using 19 channels to determine the pupillary response.

The EEG spectral index is related to the various physical and physiological states (Gastaut, 1952; Glass, 1991; Noguchi and Sakaguchi, 1999; Pfurtscheller and Da Silva, 1999; Niedermeyer, 1997; Feshchenko et al., 2001; Niedermeyer and da Silva, 2005; Cahn and Polich, 2006; Kirmizi-Alsan et al., 2006; Kisley and Cornwell, 2006; Kanayama et al., 2007; Zion-Golumbic et al., 2008; Tatum, 2014), as shown in Table 3.

Table 3 shows Comparison of EEG spectral index.

TABLE 3

| EEG | Frequency | |
|---|---|---|
| Spectral | Range | |
| Index | (Hz) | Physical and Physiological State |
| Delta | 1-4 | sleep |
| Theta | 4-8 | meditation, being sleepy, hallucinations, use |
| | | one's psychic powers, spiritual experience |
| Alpha | 8-13 | relaxation, calm state, light hypnotic, depressed |
| Beta | 13-30 | active awareness, active state, awareness, |
| | | cognitive processing, tension |
| Gamma | 30-50 | memory, learning, reminiscence, selective |
| | | concentration, highest level cognitive |
| | | processing, judgment |
| Slow-Alpha | 8-11 | relaxation, rest, predormition |
| Fast-Alpha | 11-13 | calming, concentration, creative states, a state |
| | | of tension |
| Low-Beta | 12-15 | attention, vigilance, concentration |
| Mid-Beta | 15-20 | active awareness |
| High-Beta | 20-30 | anxiety, stress, tension, mental strain |
| Mu | 9-11 | performance, observation, imagination, |
| | | empathy, mirror neuron activation |
| SMR | 12.5-15.5 | immobility, active sensory or motor areas, |
| | | attention |

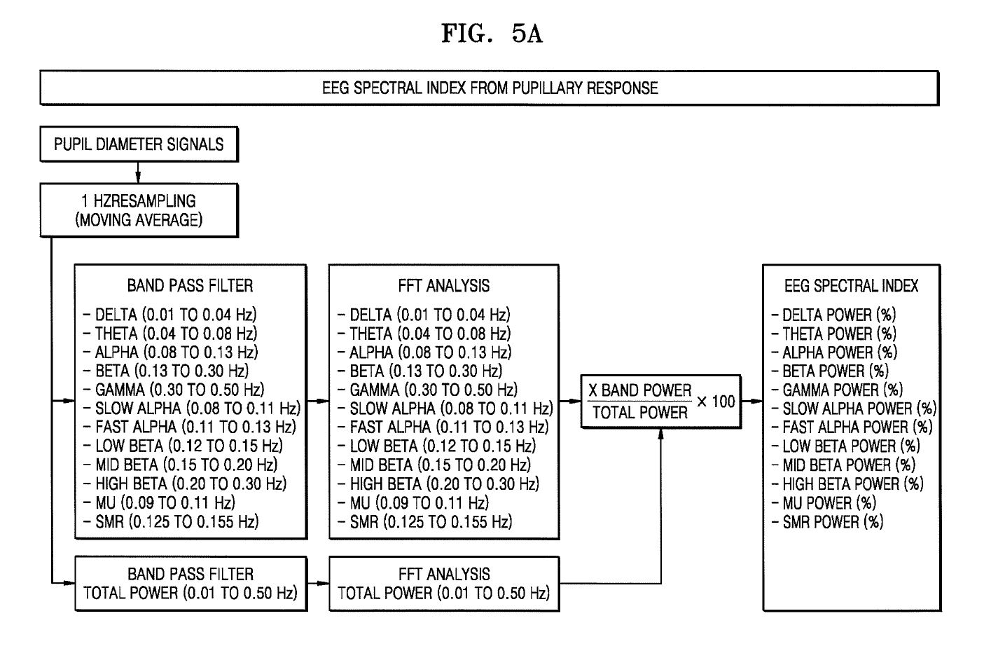

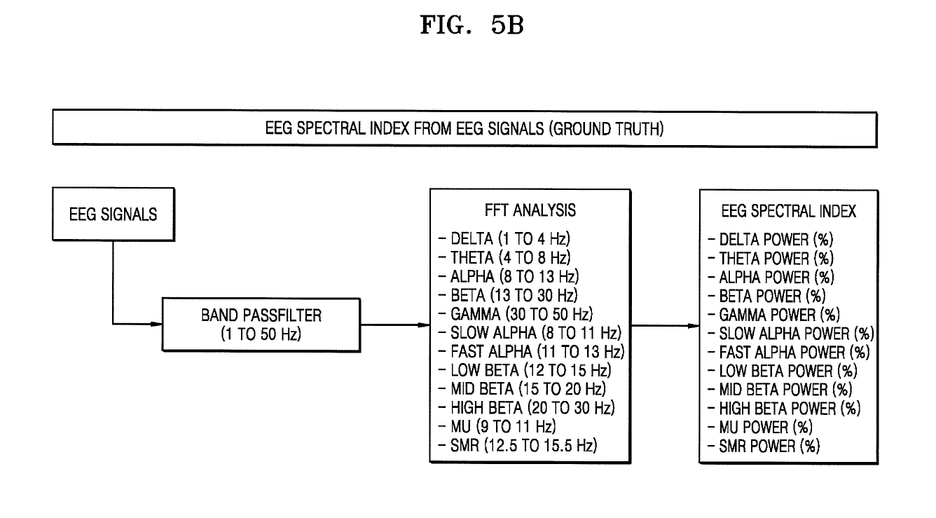

FIGS. 5A and 5B show the procedure of signal (data) processing to detect EEG spectrum index from pupillary response and EEG signals (ground truth).

Referring FIG. 5A, the re-sampled pupil diameter data at 1 Hz was filtered by the BPF of 0.01 Hz-0.50 Hz range and processed by frequency analysis to obtain following parameters: delta range of 0.01 Hz-0.04 Hz, theta range of 0.04 Hz-0.08 Hz, alpha range of 0.08 Hz-0.13 Hz, beta range of 0.13 Hz-0.30 Hz, gamma range of 0.30 Hz-0.50 Hz, slow alpha range of 0.08 Hz-0.11 Hz, fast alpha range of 0.11 Hz-0.13 Hz, low beta range of 0.12 Hz-0.15 Hz, mid beta range of 0.15 Hz-0.20 Hz, high beta range of 0.20 Hz-0.30 Hz, mu range of 0.09 Hz-0.11 Hz, SMR range of 0.125 Hz-0.155 Hz, and Total band range of 0.01 Hz-0.50 Hz.

These BPF ranges were applied by the harmonic frequency with a 1/100 resolution. The filtered signal was processed to extract each frequency band data by using frequency analysis (e.g. FFT analysis), and the total power (X power) as outputs for the each frequency band was calculated as shown in Equation 4.

$$X Power ( \% ) = \frac{X band power}{Total band power} \times 100 \\ X = δ , θ , α , β , γ , slow ( α ) , fast ( α ) , low ( β ) , \\ mid ( β ) , high ( β ) , μ , SMR \qquad < Equation 4 >$$

The outputs, that is, the powers (X power) of each frequency band, from delta to SMR, were calculated using the ratio between the total band power and EEG spectral index, as shown in Equation 4. This procedure was processed by the sliding window technique by using a window size of 180 sec and a resolution of 1 sec.

The EEG signals of ground truth were processed by using a BPF of 1 Hz-50 Hz range and the FFT analysis as shown in FIG. 5B. The EEG spectral indices obtained from the EEG signals include delta range of 1 Hz-4 Hz, theta range of 4 Hz-8 Hz, alpha range of 8 Hz-13 Hz, beta range of 13 Hz-30 Hz, gamma range of 30 Hz-50 Hz; slow alpha range of 8 Hz-11 Hz, fast alpha range of 11 Hz-13 Hz, low beta range of 12 Hz-15 Hz; mid beta range of 15 Hz-20 Hz, high beta range of 20 Hz-30 Hz, mu range of 9 Hz-11 Hz, and SMR range of 12.5 Hz-15.5 Hz.

Result

In this section, the vital signs from the cardiac time domain index, cardiac frequency domain index, EEG spectral index, and the HEP index of the test subjects were extracted from the pupillary response. These components were compared with each index from the sensor signals (i.e., ground truth) based on correlation coefficient (r) and mean error value (ME). The data was analyzed in both MNC and NMC for the test subjects.

To verify the difference of the amount movement between the two conditions of MNC and NMC, the movement data was quantitatively analyzed. The movement data was a normal distribution based on a normality test of probability-value (p) >0.05, and from an independent t-test. A Bonferroni correction was performed for the derived statistical significances (Dunnett, 1955). The statistical significance level was controlled based on the number of each individual hypothesis (i.e., α=0.05/n). The statistical significant level of the movement data sat up 0.0167 (upper body, X and Y axis in face, α=0.05/3). The effect size based on Cohen's d was also calculated to confirm practical significance. In Cohen's d, standard values of 0.10, 0.25, and 0.40 for effect size are generally regarded as small, medium, and large, respectively (Cohen, 2013).

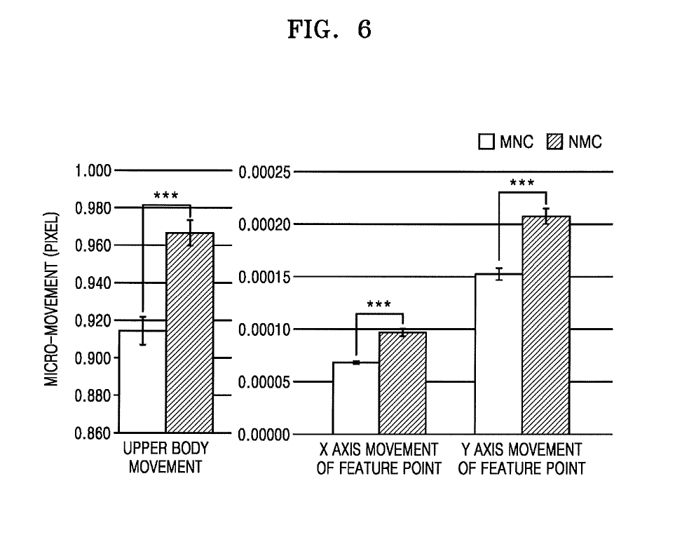

According to the analysis results, the amount of movement in MNC (upper body, X and Y axis for the face) was significantly increased compared to the NMC for the upper body (t(138)=−5.121, p=0.000, Cohen's d=1.366 with large effect size), X axis for the face (t(138)=−6.801, p=0.000, Cohen's d=1.158 with large effect size), and Y axis for the face (t(138)=−6.255, p=0.000, Cohen's d=1.118 with large effect size), as shown in FIG. 6 and Table 4.

TABLE 4

| | Movelessness Condition | | | Natural Movement Condition | | |
|---|---|---|---|---|---|---|
| Subjects | (MNC) | | | (NMC) | | |
| Subjects | Upper body | X axis | Y axis | Upper body | X axis | Y axis |
| S1 | 0.972675 | 0.000073 | 0.000158 | 1.003305 | 0.000117 | 0.000237 |
| S2 | 0.961020 | 0.000081 | 0.000170 | 1.002237 | 0.000101 | 0.000243 |
| S3 | 0.942111 | 0.000071 | 0.000206 | 0.945477 | 0.000081 | 0.000220 |
| S4 | 0.955444 | 0.000067 | 0.000189 | 0.960506 | 0.000072 | 0.000191 |
| S5 | 0.931979 | 0.000056 | 0.000106 | 0.972033 | 0.000070 | 0.000153 |
| S6 | 0.910416 | 0.000057 | 0.000103 | 0.999692 | 0.000086 | 0.000174 |
| S7 | 0.862268 | 0.000055 | 0.000216 | 0.867949 | 0.000071 | 0.000249 |
| S8 | 0.832109 | 0.000056 | 0.000182 | 0.884868 | 0.000068 | 0.000277 |
| S9 | 0.890771 | 0.000099 | 0.000188 | 0.890783 | 0.000099 | 0.000242 |
| S10 | 0.869373 | 0.000073 | 0.000168 | 0.872451 | 0.000089 | 0.000206 |
| S11 | 0.908724 | 0.000057 | 0.000128 | 0.963280 | 0.000102 | 0.000187 |
| S12 | 0.954168 | 0.000091 | 0.000180 | 0.964322 | 0.000181 | 0.000190 |
| S13 | 0.846164 | 0.000070 | 0.000144 | 0.917798 | 0.000079 | 0.000172 |
| S14 | 0.953219 | 0.000062 | 0.000116 | 1.024050 | 0.000093 | 0.000185 |
| S15 | 0.936300 | 0.000068 | 0.000202 | 0.952505 | 0.000101 | 0.000287 |
| S16 | 0.943040 | 0.000077 | 0.000220 | 0.958412 | 0.000106 | 0.000308 |
| S17 | 0.852292 | 0.000099 | 0.000199 | 0.901039 | 0.000077 | 0.000310 |
| S18 | 0.901182 | 0.000082 | 0.000278 | 0.920493 | 0.000084 | 0.000262 |
| S19 | 0.943810 | 0.000075 | 0.000156 | 0.974675 | 0.000099 | 0.000386 |
| S20 | 0.988983 | 0.000070 | 0.000162 | 1.029716 | 0.000175 | 0.000184 |
| S21 | 0.952451 | 0.000065 | 0.000102 | 1.005191 | 0.000081 | 0.000141 |
| S22 | 0.965017 | 0.000064 | 0.000099 | 0.999090 | 0.000183 | 0.000150 |
| S23 | 1.068848 | 0.000101 | 0.000200 | 1.090858 | 0.000108 | 0.000255 |
| S24 | 0.993841 | 0.000092 | 0.000184 | 1.052424 | 0.000111 | 0.000247 |
| S25 | 0.883615 | 0.000064 | 0.000258 | 0.913927 | 0.000077 | 0.000283 |
| S26 | 0.870531 | 0.000051 | 0.000221 | 0.906540 | 0.000074 | 0.000252 |
| S27 | 0.955718 | 0.000064 | 0.000126 | 0.963460 | 0.000071 | 0.000169 |
| S28 | 0.968524 | 0.000061 | 0.000142 | 0.985782 | 0.000075 | 0.000184 |
| S29 | 0.794718 | 0.000067 | 0.000119 | 0.918873 | 0.000074 | 0.000136 |
| S30 | 0.817818 | 0.000064 | 0.000105 | 0.914591 | 0.000073 | 0.000148 |
| S31 | 0.937005 | 0.000053 | 0.000138 | 0.979654 | 0.000080 | 0.000203 |
| S32 | 0.974895 | 0.000067 | 0.000204 | 1.011137 | 0.000072 | 0.000215 |
| S33 | 0.877308 | 0.000073 | 0.000134 | 0.899194 | 0.000087 | 0.000196 |
| S34 | 0.867672 | 0.000063 | 0.000127 | 0.894298 | 0.000077 | 0.000188 |
| S35 | 0.948874 | 0.000099 | 0.000182 | 0.952532 | 0.000105 | 0.000217 |
| S36 | 0.968912 | 0.000109 | 0.000217 | 1.020322 | 0.000115 | 0.000240 |
| S37 | 0.811181 | 0.000063 | 0.000204 | 0.964774 | 0.000071 | 0.000244 |
| S38 | 0.921204 | 0.000061 | 0.000160 | 0.966262 | 0.000071 | 0.000213 |
| S39 | 0.907618 | 0.000060 | 0.000151 | 0.951832 | 0.000076 | 0.000188 |
| S40 | 0.907953 | 0.000061 | 0.000169 | 0.920784 | 0.000071 | 0.000188 |
| S41 | 0.907145 | 0.000055 | 0.000151 | 0.937417 | 0.000171 | 0.000196 |
| S42 | 0.909996 | 0.000055 | 0.000163 | 0.995645 | 0.000072 | 0.000222 |
| S43 | 0.940886 | 0.000061 | 0.000137 | 0.971473 | 0.000082 | 0.000188 |
| S44 | 0.979163 | 0.000059 | 0.000127 | 1.058006 | 0.000184 | 0.000244 |
| S45 | 0.946343 | 0.000056 | 0.000109 | 1.029439 | 0.000082 | 0.000156 |
| S46 | 0.951810 | 0.000061 | 0.000154 | 0.977621 | 0.000087 | 0.000256 |
| S47 | 0.809073 | 0.000060 | 0.000147 | 0.961375 | 0.000065 | 0.000252 |
| S48 | 0.961124 | 0.000073 | 0.000176 | 0.997457 | 0.000083 | 0.000189 |
| S49 | 0.994281 | 0.000074 | 0.000172 | 1.020115 | 0.000094 | 0.000222 |
| S50 | 0.853841 | 0.000075 | 0.000194 | 0.978026 | 0.000104 | 0.000247 |
| S51 | 0.818171 | 0.000059 | 0.000168 | 0.850567 | 0.000091 | 0.000255 |
| S52 | 0.845488 | 0.000072 | 0.000134 | 0.895100 | 0.000105 | 0.000293 |
| S53 | 0.899975 | 0.000081 | 0.000150 | 0.967366 | 0.000094 | 0.000179 |
| S54 | 0.819878 | 0.000057 | 0.000106 | 0.907099 | 0.000108 | 0.000193 |
| S55 | 0.824809 | 0.000061 | 0.000119 | 0.854062 | 0.000062 | 0.000125 |
| S56 | 0.829834 | 0.000067 | 0.000126 | 0.915019 | 0.000169 | 0.000157 |
| S57 | 0.836302 | 0.000066 | 0.000126 | 0.892036 | 0.000083 | 0.000172 |
| S58 | 0.876029 | 0.000065 | 0.000155 | 0.988827 | 0.000186 | 0.000163 |
| S59 | 0.876581 | 0.000065 | 0.000149 | 0.924143 | 0.000117 | 0.000296 |
| S60 | 0.881068 | 0.000101 | 0.000252 | 1.063924 | 0.000109 | 0.000381 |
| S61 | 0.880455 | 0.000055 | 0.000093 | 1.007333 | 0.000080 | 0.000190 |
| S62 | 0.900065 | 0.000055 | 0.000087 | 1.028052 | 0.000076 | 0.000176 |
| S63 | 1.045809 | 0.000056 | 0.000102 | 1.061254 | 0.000096 | 0.000161 |
| S64 | 1.067929 | 0.000052 | 0.000105 | 1.070771 | 0.000111 | 0.000162 |
| S65 | 0.949971 | 0.000055 | 0.000101 | 1.004960 | 0.000068 | 0.000143 |
| S66 | 0.964054 | 0.000053 | 0.000093 | 1.068673 | 0.000169 | 0.000140 |
| S67 | 0.828268 | 0.000054 | 0.000082 | 0.886462 | 0.000061 | 0.000117 |
| S68 | 0.922679 | 0.000049 | 0.000079 | 0.945291 | 0.000061 | 0.000102 |
| S69 | 0.946723 | 0.000063 | 0.000112 | 1.069926 | 0.000114 | 0.000119 |
| S70 | 0.977655 | 0.000064 | 0.000113 | 0.999438 | 0.000065 | 0.000119 |
| mean | 0.914217 | 0.000067 | 0.000153 | 0.966343 | 0.000096 | 0.000208 |
| SD | 0.061596 | 0.000014 | 0.000044 | 0.057911 | 0.000033 | 0.000058 |

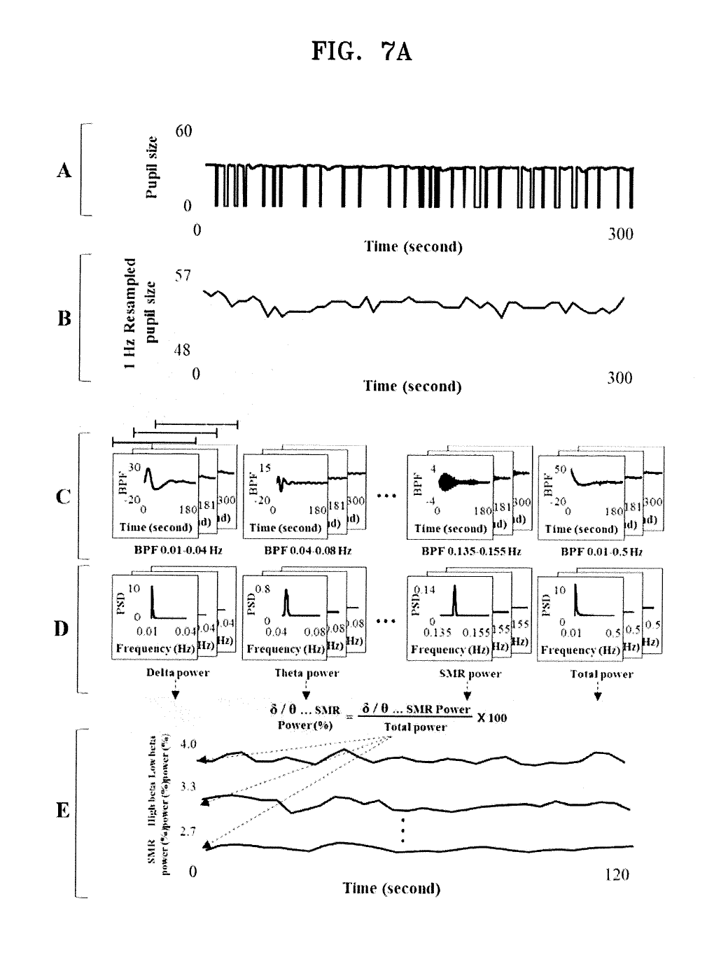

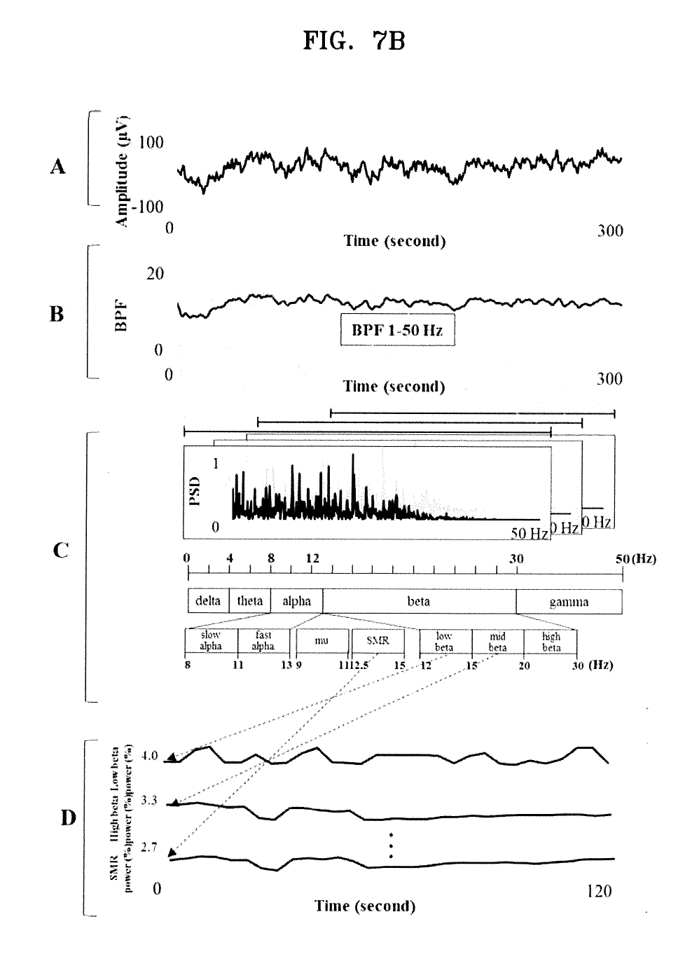

The EEG spectral index of the brain activity, as represented by delta, theta, alpha, beta, gamma, slow alpha, fast alpha, low beta, mid beta, high beta, mu, and SMR power for the 19 channel brain regions, were extracted from the pupillary response. These components were compared with the EEG spectral index from EEG signals of ground truth. The examples of EEG spectral index extraction from the pupillary response and ECG signals are shown in FIG. 7.

This exemplary study was able to determine the EEG spectral power (e.g., low beta in FP1, mid beta in FP1, SMR in FP1, beta in F3, high beta in F8, mu in C3, and gamma in P3) from the pupillary response by the entrainment of the harmonic frequency.

The EEG spectral index of brain activity ranged from 12 Hz to 15 Hz for low beta; 15 Hz to 20 Hz for mid beta; 12.5 Hz to 13.5 Hz for SMR; 13 Hz to 30 Hz for beta; 20 Hz to 30 Hz for high beta: 9 Hz to 11 Hz for mu; and 30 Hz to 50 Hz for gamma were closely connected with the circadian pupillary rhythm within the range of 0.12 Hz to 0.15 Hz; 0.15 Hz to 0.20 Hz; 0.125 Hz to 0.135 Hz; 0.13 Hz to 0.30 Hz; 0.20 Hz to 0.30 Hz; 0.09 Hz to 0.11 Hz; and 0.30 Hz to 0.50 Hz (harmonic frequency of 1/100f), respectively.

The exemplary process of extracting the EEG spectral index from the pupillary response in subjects, is shown in FIG. 7A.

(A): Signal of pupil size variation

(B): Signal re-sampled at 1 Hz based on sliding moving average technique (window size: 30 fps, resolution: 30 fps)

(C): Signals processed by BPF of each frequency band.

(D): Signals by FFT analysis

(E): Power signals as outputs of delta to SMR (0.01 Hz-0.50 Hz)

Followings are Frequency bands obtained from pupillary response.

1) delta: 0.01 Hz˜0.04 Hz

2) theta: 0.04 Hz˜0.08 Hz

3) alpha: 0.08 Hz˜0.13 Hz

4) beta: 0.13 Hz˜0.30 Hz

5) gamma: 0.30 Hz˜0.50 Hz

6) slow alpha: 0.08 Hz˜0.11 Hz

7) fast alpha: 0.11 Hz˜0.13 Hz

8) low beta: 0.12 Hz˜0.15 Hz

9) mid beta: 0.15 Hz to 0.20 Hz,

10) high beta: 0.20 Hz˜0.30 Hz

11) mu (μ): 0.09 Hz˜0.11 Hz, and

12) SMR: 0.125 Hz˜0.135 Hz) with harmonic frequency of 1/100f

The process of extracting the EEG spectral index from EEG raw data of ground truth in subjects, is shown in FIG. 7B.

(A): Raw signal of EEG (ground truth)

(B): Filtered EEG signal by BPF of 1 Hz-50 Hz

(C): Spectrum analysis and extraction powers of each frequency band (delta to SMR)

(D): Power signals (output) of each frequency band of EEG signal (ground truth)

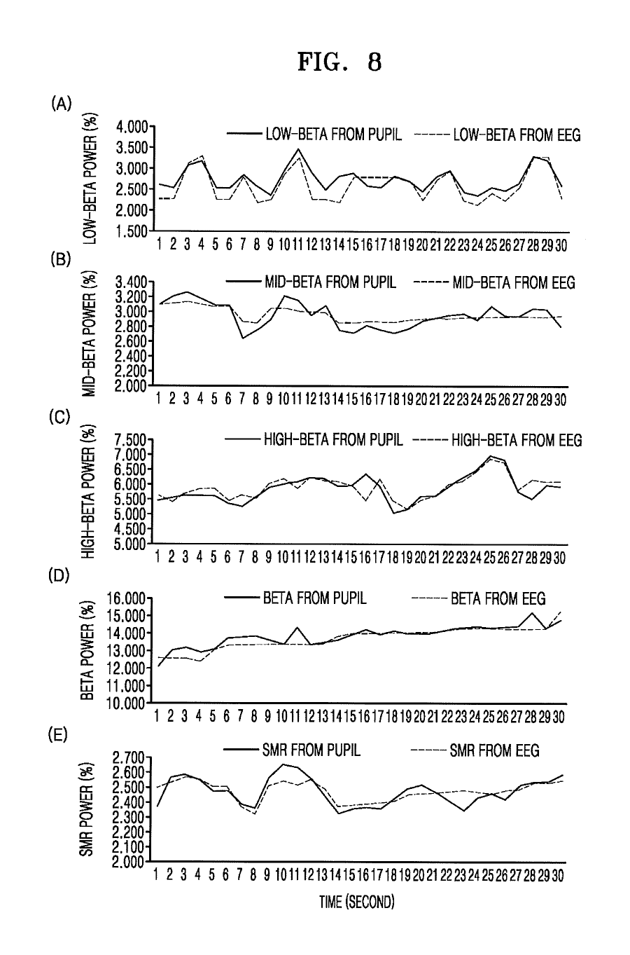

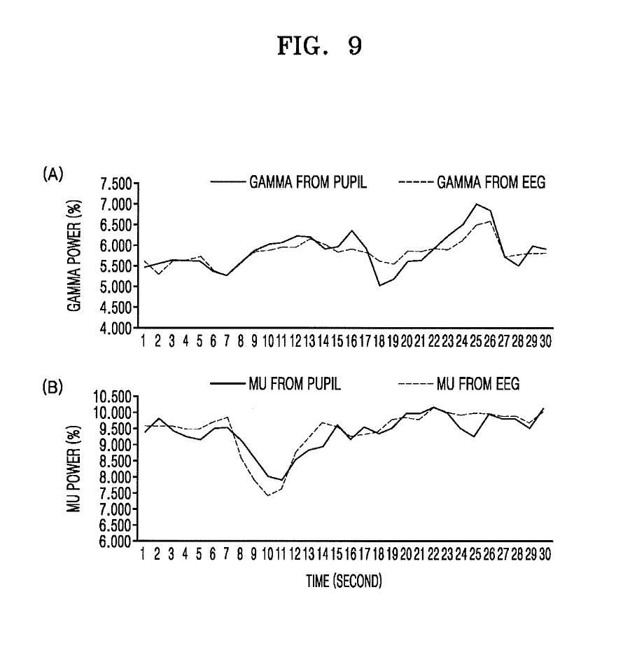

FIGS. 8 and 9 show comparison of each frequency band power between EEG spectral index from the pupillary response and EEG signal of ground truth.

In detail, FIG. 8 is an exemplary comparison graph of the EEG spectral index (frontal cortex) in MNC, wherein r=0.863, ME=0.141 for low beta in FP1, r=0.853, ME=0.004 for mid beta in FP1, r=0.857, ME=0.154 for high beta in F8, r=0.826, ME=0.052 for beta in F3, r=0.800, ME=0.002 for SMR in FP1.

In detail, FIG. 9 is an exemplary comparison graph of the EEG spectral index (parietal and central cortex) in MNC, wherein r=0.882, ME=0.039 for gamma in P4 and r=0.882, ME=0.050 for mu in C4.

When comparing the results of the ground truth in MNC, the EEG spectral index from the pupillary response indicated a strong correlation for all parameters where r=0.754±0.057 for low beta power in the FP1 region; r=0.760±0.056±for mid beta power in the FP1 region; r=0.754±0.059 for SMR power in the FP1 region; r=0.757±0.062 for beta power in the F3 region; r=0.754±0.056 for high beta power in the F8 region; r=0.762±0.055 for mu power in the C4 region; and r=0.756±0.055 for gamma power in the P4 region.

The difference between the mean error of all parameters was low where ME=0.167±0.081 for low beta power in FP1 region; ME=0.172±0.085 for mid beta power in the FP1 region; ME=0.169±0.088 for SMR power in the FP1 region; ME=0.160±0.080 for beta power in the F3 region; ME=0.178±0.081 for high beta power in the F8 region; ME=0.157±0.076 for mu power in the C4 region; and ME=0.167±0.089 for gamma power in the P4 region.

This procedure was processed using the sliding window technique where the window size was 180 s and the resolution was 1 s by using the recorded data for 300 s. The correlation and mean error were the mean value for the 70 test subjects (in one subject, N=120), as shown in Tables 4 and 5.

Table 5 shows average of correlation coefficient of EEG spectral index in MNC (N=120, p<0.01)

TABLE 5

| | Correlation coefficient | | | | | | |
|---|---|---|---|---|---|---|---|
| | low-beta | mid-beta | SMR | beta | high-beta | gamma | mu |
| Subjects | FP1 | FP1 | FP1 | F3 | F8 | P4 | C4 |
| S1 | 0.717 | 0.786 | 0.766 | 0.791 | 0.696 | 0.716 | 0.817 |
| S2 | 0.661 | 0.672 | 0.772 | 0.777 | 0.725 | 0.812 | 0.797 |
| S3 | 0.702 | 0.787 | 0.845 | 0.763 | 0.781 | 0.714 | 0.795 |
| S4 | 0.725 | 0.780 | 0.654 | 0.667 | 0.746 | 0.749 | 0.746 |
| S5 | 0.673 | 0.783 | 0.754 | 0.690 | 0.810 | 0.768 | 0.726 |
| S6 | 0.863 | 0.853 | 0.800 | 0.857 | 0.826 | 0.882 | 0.882 |
| S7 | 0.678 | 0.706 | 0.675 | 0.826 | 0.763 | 0.707 | 0.823 |
| S8 | 0.710 | 0.790 | 0.719 | 0.680 | 0.727 | 0.699 | 0.742 |
| S9 | 0.734 | 0.746 | 0.825 | 0.813 | 0.674 | 0.818 | 0.769 |
| S10 | 0.704 | 0.715 | 0.658 | 0.783 | 0.803 | 0.786 | 0.799 |
| S11 | 0.731 | 0.829 | 0.708 | 0.789 | 0.812 | 0.755 | 0.715 |
| S12 | 0.726 | 0.759 | 0.748 | 0.760 | 0.785 | 0.781 | 0.751 |
| S13 | 0.801 | 0.728 | 0.772 | 0.763 | 0.814 | 0.730 | 0.822 |
| S14 | 0.732 | 0.846 | 0.762 | 0.748 | 0.694 | 0.842 | 0.829 |
| S15 | 0.717 | 0.822 | 0.677 | 0.652 | 0.696 | 0.758 | 0.725 |
| S16 | 0.651 | 0.827 | 0.677 | 0.694 | 0.662 | 0.735 | 0.696 |
| S17 | 0.838 | 0.778 | 0.739 | 0.746 | 0.678 | 0.760 | 0.694 |
| S18 | 0.780 | 0.791 | 0.651 | 0.830 | 0.674 | 0.722 | 0.715 |
| S19 | 0.792 | 0.777 | 0.661 | 0.728 | 0.811 | 0.794 | 0.699 |
| S20 | 0.752 | 0.767 | 0.748 | 0.792 | 0.739 | 0.829 | 0.849 |
| S21 | 0.747 | 0.806 | 0.743 | 0.806 | 0.678 | 0.751 | 0.726 |
| S22 | 0.678 | 0.719 | 0.669 | 0.702 | 0.714 | 0.733 | 0.753 |
| S23 | 0.696 | 0.768 | 0.779 | 0.827 | 0.685 | 0.797 | 0.790 |
| S24 | 0.836 | 0.755 | 0.761 | 0.710 | 0.720 | 0.802 | 0.668 |
| S25 | 0.669 | 0.747 | 0.821 | 0.723 | 0.703 | 0.740 | 0.702 |
| S26 | 0.832 | 0.662 | 0.825 | 0.740 | 0.689 | 0.826 | 0.752 |
| S27 | 0.710 | 0.691 | 0.824 | 0.814 | 0.655 | 0.756 | 0.788 |
| S28 | 0.675 | 0.747 | 0.792 | 0.812 | 0.801 | 0.808 | 0.786 |
| S29 | 0.846 | 0.713 | 0.704 | 0.761 | 0.818 | 0.786 | 0.714 |
| S30 | 0.787 | 0.664 | 0.701 | 0.796 | 0.795 | 0.739 | 0.774 |
| S31 | 0.842 | 0.753 | 0.789 | 0.810 | 0.839 | 0.667 | 0.751 |
| S32 | 0.689 | 0.760 | 0.846 | 0.661 | 0.711 | 0.660 | 0.762 |
| S33 | 0.754 | 0.758 | 0.830 | 0.739 | 0.693 | 0.806 | 0.686 |
| S34 | 0.802 | 0.798 | 0.831 | 0.707 | 0.796 | 0.773 | 0.840 |
| S35 | 0.704 | 0.817 | 0.742 | 0.758 | 0.704 | 0.770 | 0.722 |
| S36 | 0.832 | 0.752 | 0.762 | 0.705 | 0.705 | 0.791 | 0.686 |
| S37 | 0.774 | 0.680 | 0.795 | 0.825 | 0.800 | 0.735 | 0.800 |
| S38 | 0.708 | 0.664 | 0.763 | 0.676 | 0.770 | 0.740 | 0.680 |
| S39 | 0.687 | 0.720 | 0.792 | 0.816 | 0.728 | 0.656 | 0.715 |
| S40 | 0.717 | 0.846 | 0.662 | 0.759 | 0.815 | 0.747 | 0.796 |
| S41 | 0.708 | 0.747 | 0.849 | 0.811 | 0.786 | 0.793 | 0.731 |
| S42 | 0.862 | 0.803 | 0.840 | 0.882 | 0.838 | 0.866 | 0.868 |
| S43 | 0.667 | 0.725 | 0.840 | 0.833 | 0.680 | 0.698 | 0.815 |
| S44 | 0.800 | 0.678 | 0.813 | 0.698 | 0.701 | 0.809 | 0.749 |
| S45 | 0.679 | 0.678 | 0.748 | 0.827 | 0.776 | 0.846 | 0.738 |
| S46 | 0.770 | 0.655 | 0.661 | 0.656 | 0.655 | 0.845 | 0.814 |
| S47 | 0.779 | 0.841 | 0.668 | 0.815 | 0.808 | 0.687 | 0.750 |
| S48 | 0.744 | 0.769 | 0.725 | 0.679 | 0.845 | 0.659 | 0.667 |
| S49 | 0.704 | 0.773 | 0.808 | 0.674 | 0.728 | 0.734 | 0.671 |
| S50 | 0.675 | 0.769 | 0.652 | 0.661 | 0.727 | 0.704 | 0.778 |
| S51 | 0.838 | 0.791 | 0.735 | 0.683 | 0.778 | 0.720 | 0.765 |
| S52 | 0.829 | 0.759 | 0.715 | 0.832 | 0.819 | 0.773 | 0.684 |
| S53 | 0.819 | 0.818 | 0.824 | 0.850 | 0.804 | 0.773 | 0.664 |
| S54 | 0.736 | 0.817 | 0.660 | 0.660 | 0.820 | 0.811 | 0.767 |
| S55 | 0.745 | 0.757 | 0.800 | 0.833 | 0.765 | 0.742 | 0.821 |
| S56 | 0.766 | 0.825 | 0.704 | 0.835 | 0.740 | 0.763 | 0.658 |
| S57 | 0.827 | 0.881 | 0.710 | 0.750 | 0.792 | 0.795 | 0.705 |
| S58 | 0.725 | 0.757 | 0.815 | 0.839 | 0.763 | 0.696 | 0.795 |
| S59 | 0.736 | 0.662 | 0.809 | 0.656 | 0.705 | 0.702 | 0.727 |
| S60 | 0.755 | 0.771 | 0.791 | 0.680 | 0.735 | 0.662 | 0.792 |
| S61 | 0.741 | 0.704 | 0.776 | 0.771 | 0.856 | 0.870 | 0.843 |
| S62 | 0.831 | 0.735 | 0.714 | 0.731 | 0.762 | 0.749 | 0.739 |
| S63 | 0.823 | 0.653 | 0.817 | 0.783 | 0.837 | 0.829 | 0.820 |
| S64 | 0.806 | 0.859 | 0.735 | 0.732 | 0.750 | 0.847 | 0.802 |
| S65 | 0.763 | 0.737 | 0.719 | 0.673 | 0.841 | 0.715 | 0.762 |
| S66 | 0.792 | 0.845 | 0.760 | 0.776 | 0.741 | 0.812 | 0.662 |
| S67 | 0.794 | 0.846 | 0.728 | 0.724 | 0.658 | 0.755 | 0.833 |
| S68 | 0.804 | 0.717 | 0.804 | 0.764 | 0.737 | 0.718 | 0.665 |
| S69 | 0.777 | 0.747 | 0.693 | 0.842 | 0.778 | 0.683 | 0.763 |
| S70 | 0.799 | 0.722 | 0.830 | 0.739 | 0.800 | 0.822 | 0.812 |
| mean | 0.754 | 0.760 | 0.754 | 0.757 | 0.754 | 0.762 | 0.756 |
| SD | 0.057 | 0.056 | 0.059 | 0.062 | 0.056 | 0.055 | 0.056 |

Table 6 shows average of mean error of EEG spectral index in MNC (N=120)

TABLE 6

| | Mean error | | | | | | |
|---|---|---|---|---|---|---|---|
| | low-beta | mid-beta | SMR | beta | high-beta | gamma | mu |
| Subjects | FP1 | FP1 | FP1 | F3 | F8 | P4 | C4 |
| S1 | 0.211 | 0.212 | 0.227 | 0.161 | 0.101 | 0.268 | 0.108 |
| S2 | 0.035 | 0.148 | 0.238 | 0.249 | 0.297 | 0.224 | 0.153 |
| S3 | 0.157 | 0.052 | 0.187 | 0.362 | 0.145 | 0.081 | 0.072 |
| S4 | 0.075 | 0.106 | 0.180 | 0.088 | 0.149 | 0.085 | 0.029 |
| S5 | 0.074 | 0.182 | 0.081 | 0.045 | 0.026 | 0.220 | 0.067 |
| S6 | 0.244 | 0.181 | 0.250 | 0.075 | 0.287 | 0.197 | 0.232 |
| S7 | 0.069 | 0.292 | 0.121 | 0.101 | 0.297 | 0.187 | 0.289 |
| S8 | 0.176 | 0.091 | 0.115 | 0.234 | 0.250 | 0.102 | 0.208 |
| S9 | 0.110 | 0.158 | 0.168 | 0.079 | 0.100 | 0.035 | 0.174 |
| S10 | 0.197 | 0.141 | 0.032 | 0.035 | 0.246 | 0.152 | 0.076 |
| S11 | 0.183 | 0.160 | 0.222 | 0.265 | 0.215 | 0.132 | 0.081 |
| S12 | 0.077 | 0.223 | 0.098 | 0.101 | 0.060 | 0.048 | 0.231 |
| S13 | 0.075 | 0.193 | 0.273 | 0.156 | 0.157 | 0.160 | 0.199 |
| S14 | 0.282 | 0.254 | 0.157 | 0.144 | 0.106 | 0.075 | 0.080 |
| S15 | 0.173 | 0.047 | 0.246 | 0.246 | 0.233 | 0.288 | 0.102 |
| S16 | 0.452 | 0.217 | 0.449 | 0.217 | 0.218 | 0.119 | 0.106 |
| S17 | 0.254 | 0.200 | 0.094 | 0.133 | 0.288 | 0.221 | 0.240 |
| S18 | 0.121 | 0.135 | 0.105 | 0.211 | 0.176 | 0.214 | 0.036 |
| S19 | 0.144 | 0.127 | 0.278 | 0.210 | 0.210 | 0.185 | 0.133 |
| S20 | 0.235 | 0.262 | 0.300 | 0.128 | 0.459 | 0.276 | 0.278 |
| S21 | 0.165 | 0.094 | 0.094 | 0.076 | 0.214 | 0.270 | 0.263 |
| S22 | 0.103 | 0.046 | 0.042 | 0.143 | 0.184 | 0.034 | 0.115 |
| S23 | 0.087 | 0.181 | 0.138 | 0.158 | 0.155 | 0.154 | 0.030 |
| S24 | 0.167 | 0.299 | 0.133 | 0.059 | 0.119 | 0.174 | 0.204 |
| S25 | 0.071 | 0.240 | 0.023 | 0.063 | 0.035 | 0.175 | 0.126 |
| S26 | 0.053 | 0.031 | 0.149 | 0.294 | 0.256 | 0.025 | 0.196 |
| S27 | 0.223 | 0.295 | 0.093 | 0.237 | 0.171 | 0.149 | 0.218 |
| S28 | 0.265 | 0.121 | 0.269 | 0.087 | 0.190 | 0.080 | 0.143 |
| S29 | 0.091 | 0.049 | 0.208 | 0.236 | 0.252 | 0.251 | 0.109 |
| S30 | 0.091 | 0.143 | 0.186 | 0.099 | 0.235 | 0.210 | 0.254 |
| S31 | 0.218 | 0.238 | 0.238 | 0.168 | 0.152 | 0.043 | 0.213 |
| S32 | 0.124 | 0.297 | 0.207 | 0.132 | 0.158 | 0.293 | 0.174 |
| S33 | 0.124 | 0.207 | 0.027 | 0.209 | 0.151 | 0.204 | 0.214 |
| S34 | 0.066 | 0.187 | 0.282 | 0.095 | 0.108 | 0.136 | 0.299 |
| S35 | 0.146 | 0.252 | 0.281 | 0.243 | 0.071 | 0.155 | 0.027 |
| S36 | 0.153 | 0.377 | 0.123 | 0.213 | 0.289 | 0.156 | 0.220 |
| S37 | 0.198 | 0.159 | 0.050 | 0.210 | 0.054 | 0.110 | 0.285 |
| S38 | 0.279 | 0.063 | 0.261 | 0.202 | 0.262 | 0.156 | 0.188 |
| S39 | 0.269 | 0.152 | 0.295 | 0.125 | 0.255 | 0.203 | 0.235 |
| S40 | 0.251 | 0.210 | 0.053 | 0.073 | 0.133 | 0.105 | 0.106 |
| S41 | 0.135 | 0.267 | 0.331 | 0.273 | 0.235 | 0.208 | 0.073 |
| S42 | 0.259 | 0.124 | 0.180 | 0.033 | 0.067 | 0.234 | 0.107 |
| S43 | 0.274 | 0.069 | 0.088 | 0.218 | 0.242 | 0.216 | 0.230 |
| S44 | 0.240 | 0.286 | 0.090 | 0.122 | 0.225 | 0.135 | 0.129 |
| S45 | 0.136 | 0.202 | 0.180 | 0.137 | 0.254 | 0.074 | 0.193 |
| S46 | 0.237 | 0.210 | 0.222 | 0.237 | 0.247 | 0.276 | 0.289 |
| S47 | 0.113 | 0.098 | 0.081 | 0.040 | 0.221 | 0.220 | 0.278 |
| S48 | 0.093 | 0.350 | 0.028 | 0.290 | 0.091 | 0.092 | 0.123 |
| S49 | 0.229 | 0.197 | 0.045 | 0.088 | 0.262 | 0.079 | 0.443 |
| S50 | 0.077 | 0.081 | 0.229 | 0.045 | 0.095 | 0.289 | 0.109 |
| S51 | 0.167 | 0.091 | 0.242 | 0.068 | 0.082 | 0.034 | 0.159 |
| S52 | 0.064 | 0.284 | 0.168 | 0.026 | 0.190 | 0.111 | 0.241 |
| S53 | 0.266 | 0.132 | 0.215 | 0.208 | 0.144 | 0.163 | 0.284 |
| S54 | 0.176 | 0.061 | 0.323 | 0.222 | 0.043 | 0.078 | 0.022 |
| S55 | 0.087 | 0.248 | 0.177 | 0.093 | 0.092 | 0.123 | 0.280 |
| S56 | 0.094 | 0.236 | 0.116 | 0.216 | 0.242 | 0.166 | 0.024 |
| S57 | 0.070 | 0.022 | 0.257 | 0.225 | 0.111 | 0.074 | 0.083 |
| S58 | 0.256 | 0.273 | 0.073 | 0.262 | 0.192 | 0.263 | 0.264 |
| S59 | 0.295 | 0.080 | 0.102 | 0.197 | 0.073 | 0.184 | 0.213 |
| S60 | 0.191 | 0.217 | 0.184 | 0.204 | 0.183 | 0.249 | 0.272 |
| S61 | 0.082 | 0.123 | 0.253 | 0.250 | 0.176 | 0.045 | 0.149 |
| S62 | 0.236 | 0.091 | 0.121 | 0.120 | 0.158 | 0.074 | 0.069 |
| S63 | 0.048 | 0.145 | 0.066 | 0.214 | 0.225 | 0.073 | 0.282 |
| S64 | 0.117 | 0.049 | 0.130 | 0.085 | 0.150 | 0.281 | 0.043 |
| S65 | 0.126 | 0.297 | 0.171 | 0.204 | 0.082 | 0.218 | 0.262 |
| S66 | 0.243 | 0.044 | 0.136 | 0.186 | 0.290 | 0.159 | 0.134 |
| S67 | 0.189 | 0.289 | 0.235 | 0.226 | 0.197 | 0.083 | 0.176 |
| S68 | 0.230 | 0.202 | 0.078 | 0.266 | 0.127 | 0.235 | 0.069 |
| S69 | 0.167 | 0.163 | 0.156 | 0.061 | 0.084 | 0.181 | 0.124 |
| S70 | 0.293 | 0.100 | 0.198 | 0.038 | 0.208 | 0.051 | 0.031 |
| mean | 0.167 | 0.172 | 0.169 | 0.160 | 0.178 | 0.157 | 0.167 |
| SD | 0.081 | 0.085 | 0.088 | 0.080 | 0.081 | 0.076 | 0.089 |

The correlation and mean error matrix table between brain regions and EEG frequency ranges is shown in Tables 7 and 8. Low beta, mid beta, and SMR power from the pupillary response were strongly correlated and had little difference with the EEG band power in the FP1 and FP2 regions (r>0.5, ME<1).

Beta power from the pupillary response was strongly correlated, and had little difference, with EEG band power in the F3, F4, and Fz brain regions (r>0.5, ME<1). High beta power from the pupillary response was strongly correlated, and had little difference, with EEG band power in the F7 and F8 brain regions (r>0.5, ME<1). Mu power from the pupillary response was strongly correlated, and had little difference, with EEG band power in the C3, C4, and Cz brain regions (r>0.5, ME<1).

Gamma power from the pupillary response was strongly correlated, and had little difference, with EEG band power in the P3 and P4 brain regions (r>0.5, ME<1). Other brain regions and frequency ranges were a low correlation and indicated a large difference (r<0.5, ME>1). Low beta, mid beta, SMR, beta, high beta, mu, and gamma were the higher correlations and had very little differences (r>0.7, ME<0.2) with FP1, FP1, FP1, F3, F8, C4, and P4, respectively.

Table 6 shows average of correlation matrix between brain regions and EEG frequency ranges in MNC (dark grey shade r>0.7, light grey shade r>0.5).

Table 8 shows average of mean error matrix between brain regions and EEG frequency ranges in MNC (dark grey shade ME>0.2, light grey shade ME>1).

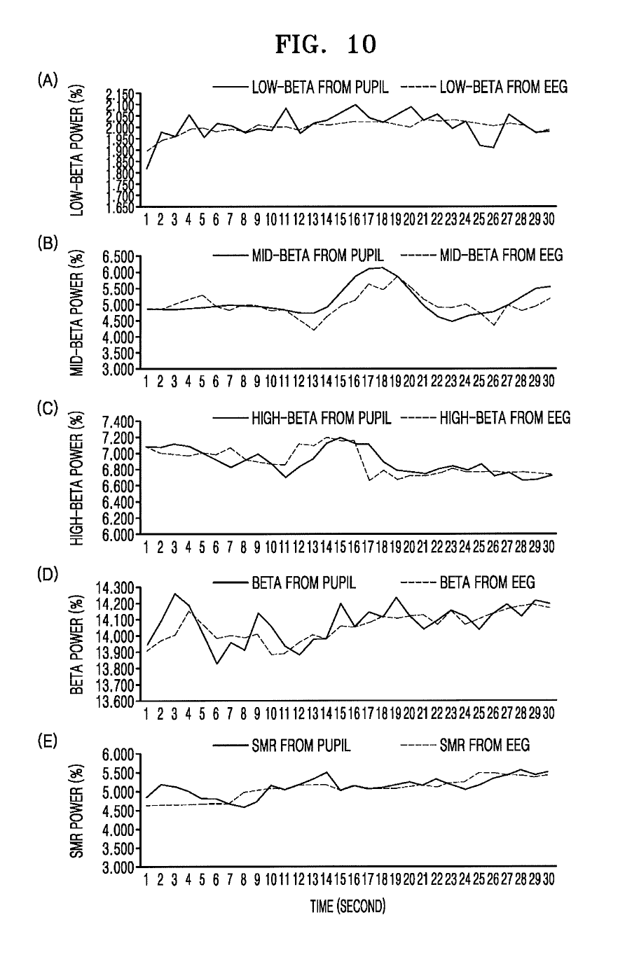

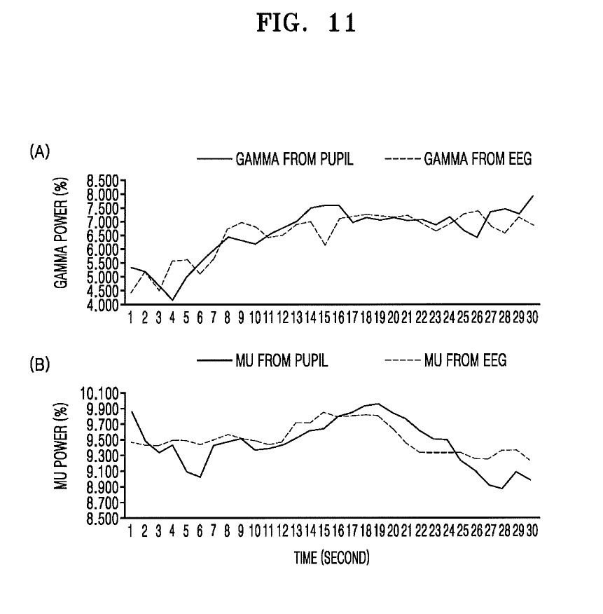

The example of extracting the EEG spectral index from the pupillary response and ECG signals for the subjects was shown in FIGS. 10 and 11.

FIG. 10 shows comparison examples of the EEG spectral index (frontal cortex) in NMC.

r=0.634, ME=0.006 for low beta in FP1

r=0.688, ME=0.106 for mid beta in FP1

r=0.656, ME=0.004 for high beta in F8

r=0.639, ME=0.020 for beta in F3

r=0.677, ME=0.055 for SMR in FP1

FIG. 11 shows comparison examples of the EEG spectral index (parietal and central cortex) in NMC.

r=0.712, ME=0.065 for gamma in P4

r=0.714, ME=0.053 for mu in C4

When comparing the results with ground truth in NMC, the EEG spectral index from pupillary response indicated a strong correlation for all parameters where r=0.642±0.057 for low beta power in the FP1 region; r=0.656±0.056 for mid beta power in the FP1 region; r=0.646±0.063 for SMR power in the FP1 region; r=0.662±0.056 for beta power in the F3 region; r=0.648±0.055 for high beta power in the F8 region; r=0.650±0.054 for mu power in the C4 region; and r=0.641±0.059 for gamma power in the P4 region.

The difference between the mean error was of all parameters was low with ME=0.494±0.196 for low beta power in the FP1 region; ME=0.472±0.180 for mid beta power in the FP1 region; ME=0.495±0.198 for SMR power in the FP1 region; ME=0.483±0.180 for beta power in the F3 region; ME=0.476±0.193 for high beta power in the F8 region; ME=0.483±0.198 for mu power in the C4 region; and ME=0.488±0.177 for gamma power in the P4 region.

This procedure was processed by the sliding window technique, where the window size was 180 s and the resolution was 1 s by using recorded data for 300 s. The correlation and mean error were the mean value for the test 70 subjects (in one subject, N=120), as shown in Tables 9 and 10.

Table 9 shows average of correlation coefficient of EEG spectral index in MNC (N=120, p<0.01).

TABLE 9

| | Correlation coefficient | | | | | | |
|---|---|---|---|---|---|---|---|
| | low-beta | mid-beta | SMR | beta | high-beta | gamma | mu |
| Subjects | FP1 | FP1 | FP1 | F3 | F8 | P4 | C4 |
| S1 | 0.575 | 0.575 | 0.574 | 0.717 | 0.708 | 0.594 | 0.690 |
| S2 | 0.672 | 0.580 | 0.750 | 0.682 | 0.594 | 0.704 | 0.726 |
| S3 | 0.687 | 0.657 | 0.664 | 0.731 | 0.607 | 0.726 | 0.685 |
| S4 | 0.578 | 0.742 | 0.597 | 0.660 | 0.601 | 0.561 | 0.565 |
| S5 | 0.625 | 0.595 | 0.695 | 0.703 | 0.607 | 0.663 | 0.618 |
| S6 | 0.634 | 0.688 | 0.677 | 0.639 | 0.656 | 0.712 | 0.714 |
| S7 | 0.617 | 0.741 | 0.749 | 0.571 | 0.623 | 0.695 | 0.605 |
| S8 | 0.602 | 0.563 | 0.666 | 0.569 | 0.586 | 0.730 | 0.583 |
| S9 | 0.707 | 0.603 | 0.646 | 0.742 | 0.558 | 0.602 | 0.581 |
| S10 | 0.656 | 0.678 | 0.553 | 0.728 | 0.713 | 0.720 | 0.660 |
| S11 | 0.555 | 0.683 | 0.553 | 0.647 | 0.721 | 0.641 | 0.740 |
| S12 | 0.616 | 0.651 | 0.576 | 0.726 | 0.706 | 0.587 | 0.606 |
| S13 | 0.667 | 0.623 | 0.570 | 0.603 | 0.672 | 0.728 | 0.616 |
| S14 | 0.739 | 0.742 | 0.551 | 0.606 | 0.692 | 0.610 | 0.674 |
| S15 | 0.593 | 0.674 | 0.734 | 0.688 | 0.576 | 0.571 | 0.603 |
| S16 | 0.609 | 0.574 | 0.633 | 0.686 | 0.684 | 0.691 | 0.554 |
| S17 | 0.581 | 0.593 | 0.749 | 0.674 | 0.555 | 0.655 | 0.577 |
| S18 | 0.595 | 0.649 | 0.658 | 0.678 | 0.572 | 0.568 | 0.590 |
| S19 | 0.673 | 0.748 | 0.729 | 0.737 | 0.699 | 0.708 | 0.749 |
| S20 | 0.691 | 0.729 | 0.620 | 0.615 | 0.582 | 0.599 | 0.618 |
| S21 | 0.633 | 0.554 | 0.675 | 0.604 | 0.638 | 0.674 | 0.592 |
| S22 | 0.569 | 0.720 | 0.624 | 0.642 | 0.646 | 0.606 | 0.616 |
| S23 | 0.559 | 0.557 | 0.637 | 0.627 | 0.649 | 0.621 | 0.710 |
| S24 | 0.732 | 0.659 | 0.643 | 0.639 | 0.690 | 0.697 | 0.669 |
| S25 | 0.567 | 0.707 | 0.628 | 0.735 | 0.557 | 0.735 | 0.639 |
| S26 | 0.675 | 0.654 | 0.573 | 0.747 | 0.743 | 0.722 | 0.714 |
| S27 | 0.642 | 0.587 | 0.733 | 0.705 | 0.611 | 0.694 | 0.555 |
| S28 | 0.565 | 0.673 | 0.686 | 0.703 | 0.612 | 0.568 | 0.626 |
| S29 | 0.631 | 0.579 | 0.645 | 0.669 | 0.696 | 0.679 | 0.591 |
| S30 | 0.714 | 0.644 | 0.566 | 0.730 | 0.618 | 0.597 | 0.610 |
| S31 | 0.646 | 0.588 | 0.568 | 0.597 | 0.660 | 0.572 | 0.592 |
| S32 | 0.554 | 0.668 | 0.646 | 0.724 | 0.634 | 0.691 | 0.655 |
| S33 | 0.595 | 0.689 | 0.736 | 0.578 | 0.744 | 0.624 | 0.600 |
| S34 | 0.617 | 0.707 | 0.611 | 0.704 | 0.722 | 0.618 | 0.745 |
| S35 | 0.604 | 0.695 | 0.743 | 0.621 | 0.695 | 0.590 | 0.706 |
| S36 | 0.725 | 0.717 | 0.557 | 0.551 | 0.555 | 0.617 | 0.709 |
| S37 | 0.695 | 0.594 | 0.627 | 0.691 | 0.615 | 0.613 | 0.648 |
| S38 | 0.657 | 0.667 | 0.689 | 0.710 | 0.599 | 0.659 | 0.617 |
| S39 | 0.620 | 0.691 | 0.556 | 0.665 | 0.739 | 0.574 | 0.573 |
| S40 | 0.592 | 0.619 | 0.737 | 0.698 | 0.601 | 0.664 | 0.562 |
| S41 | 0.731 | 0.700 | 0.744 | 0.576 | 0.589 | 0.701 | 0.621 |
| S42 | 0.670 | 0.640 | 0.644 | 0.683 | 0.702 | 0.706 | 0.722 |
| S43 | 0.654 | 0.694 | 0.597 | 0.692 | 0.652 | 0.612 | 0.593 |
| S44 | 0.728 | 0.721 | 0.743 | 0.716 | 0.588 | 0.676 | 0.677 |
| S45 | 0.645 | 0.698 | 0.614 | 0.681 | 0.589 | 0.595 | 0.668 |
| S46 | 0.602 | 0.720 | 0.739 | 0.731 | 0.742 | 0.715 | 0.579 |
| S47 | 0.659 | 0.597 | 0.646 | 0.730 | 0.645 | 0.629 | 0.555 |
| S48 | 0.611 | 0.715 | 0.734 | 0.595 | 0.722 | 0.730 | 0.724 |
| S49 | 0.596 | 0.727 | 0.577 | 0.731 | 0.672 | 0.629 | 0.645 |
| S50 | 0.742 | 0.563 | 0.564 | 0.608 | 0.714 | 0.604 | 0.620 |
| S51 | 0.622 | 0.614 | 0.670 | 0.624 | 0.649 | 0.610 | 0.553 |
| S52 | 0.736 | 0.663 | 0.723 | 0.732 | 0.726 | 0.598 | 0.572 |
| S53 | 0.559 | 0.734 | 0.633 | 0.556 | 0.587 | 0.654 | 0.588 |
| S54 | 0.739 | 0.574 | 0.657 | 0.557 | 0.605 | 0.606 | 0.707 |
| S55 | 0.729 | 0.691 | 0.624 | 0.651 | 0.633 | 0.685 | 0.634 |
| S56 | 0.566 | 0.702 | 0.618 | 0.565 | 0.691 | 0.559 | 0.718 |
| S57 | 0.639 | 0.643 | 0.596 | 0.659 | 0.655 | 0.610 | 0.724 |
| S58 | 0.615 | 0.645 | 0.554 | 0.640 | 0.675 | 0.679 | 0.678 |
| S59 | 0.744 | 0.674 | 0.644 | 0.557 | 0.738 | 0.618 | 0.585 |
| S60 | 0.668 | 0.669 | 0.745 | 0.667 | 0.643 | 0.683 | 0.748 |
| S61 | 0.652 | 0.561 | 0.552 | 0.658 | 0.724 | 0.675 | 0.746 |
| S62 | 0.584 | 0.637 | 0.627 | 0.669 | 0.606 | 0.737 | 0.576 |
| S63 | 0.740 | 0.603 | 0.612 | 0.699 | 0.742 | 0.744 | 0.594 |
| S64 | 0.716 | 0.738 | 0.682 | 0.743 | 0.617 | 0.622 | 0.584 |
| S65 | 0.639 | 0.658 | 0.567 | 0.687 | 0.617 | 0.721 | 0.698 |
| S66 | 0.571 | 0.711 | 0.588 | 0.635 | 0.616 | 0.689 | 0.642 |
| S67 | 0.702 | 0.674 | 0.677 | 0588 | 0.567 | 0.554 | 0.721 |
| S68 | 0.596 | 0.559 | 0.651 | 0.600 | 0.620 | 0.656 | 0.640 |
| S69 | 0.568 | 0.645 | 0.688 | 0.694 | 0.656 | 0.631 | 0.693 |
| S70 | 0.628 | 0.661 | 0.680 | 0.673 | 0.652 | 0.663 | 0.589 |
| mean | 0.642 | 0.656 | 0.646 | 0.662 | 0.648 | 0.650 | 0.641 |
| SD | 0.057 | 0.056 | 0.063 | 0.056 | 0.055 | 0.054 | 0.059 |

Table 10 shows average of mean error of EEG spectral index in NMC (N=120).

TABLE 10

| | Mean error | | | | | | |
|---|---|---|---|---|---|---|---|
| | low-beta | mid-beta | SMR | beta | high-beta | gamma | mu |
| Subjects | FP1 | FP1 | FP1 | F3 | F8 | P4 | C4 |
| S1 | 0.498 | 0.521 | 0.653 | 0.330 | 0.745 | 0.546 | 0.204 |
| S2 | 0.442 | 0.737 | 0.599 | 0.558 | 0.449 | 0.219 | 0.495 |
| S3 | 0.462 | 0.556 | 0.574 | 0.520 | 0.557 | 0.765 | 0.723 |
| S4 | 0.272 | 0.655 | 0.500 | 0.431 | 0.380 | 0.469 | 0.490 |
| S5 | 0.616 | 0.472 | 0.418 | 0.590 | 0.617 | 0.387 | 0.221 |
| S6 | 0.006 | 0.106 | 0.055 | 0.002 | 0.004 | 0.065 | 0.053 |
| S7 | 0.795 | 0.566 | 0.293 | 0.792 | 0.648 | 0.769 | 0.446 |
| S8 | 0.587 | 0.532 | 0.564 | 0.248 | 0.260 | 0.767 | 0.227 |
| S9 | 0.396 | 0.336 | 0.579 | 0.788 | 0.643 | 0.222 | 0.652 |
| S10 | 0.412 | 0.310 | 0.380 | 0.447 | 0.645 | 0.316 | 0.548 |
| S11 | 0.216 | 0.467 | 0.643 | 0.386 | 0.361 | 0.710 | 0.258 |
| S12 | 0.325 | 0.487 | 0.642 | 0.796 | 0.678 | 0.577 | 0.401 |
| S13 | 0.724 | 0.700 | 0.594 | 0.200 | 0.623 | 0.642 | 0.308 |
| S14 | 0.411 | 0.458 | 0.538 | 0.361 | 0.519 | 0.295 | 0.275 |
| S15 | 0.289 | 0.414 | 0.706 | 0.728 | 0.649 | 0.467 | 0.390 |
| S16 | 0.650 | 0.330 | 0.752 | 0.632 | 0.756 | 0.634 | 0.362 |
| S17 | 0.693 | 0.234 | 0.675 | 0.485 | 0.633 | 0.735 | 0.739 |
| S18 | 0.450 | 0.637 | 0.768 | 0.521 | 0.699 | 0.361 | 0.592 |
| S19 | 0.287 | 0.218 | 0.705 | 0.528 | 0.365 | 0.752 | 0.500 |
| S20 | 0.753 | 0.637 | 0.499 | 0.526 | 0.379 | 0.393 | 0.685 |
| S21 | 0.595 | 0.539 | 0.559 | 0.229 | 0.535 | 0.713 | 0.743 |
| S22 | 0.300 | 0.699 | 0.736 | 0.691 | 0.458 | 0.793 | 0.791 |
| S23 | 0.479 | 0.514 | 0.691 | 0.377 | 0.346 | 0.792 | 0.667 |
| S24 | 0.773 | 0.235 | 0.522 | 0.250 | 0.700 | 0.786 | 0.447 |
| S25 | 0.234 | 0.251 | 0.644 | 0.342 | 0.679 | 0.724 | 0.457 |
| S26 | 0.257 | 0.253 | 0.708 | 0.723 | 0.762 | 0.480 | 0.534 |
| S27 | 0.799 | 0.383 | 0.351 | 0.362 | 0.263 | 0.656 | 0.589 |
| S28 | 0.684 | 0.255 | 0.314 | 0.751 | 0.273 | 0.597 | 0.453 |
| S29 | 0.502 | 0.631 | 0.369 | 0.202 | 0.520 | 0.538 | 0.405 |
| S30 | 0.752 | 0.521 | 0.407 | 0.305 | 0.746 | 0.412 | 0.793 |
| S31 | 0.201 | 0.749 | 0.207 | 0.743 | 0.575 | 0.287 | 0.474 |
| S32 | 0.444 | 0.697 | 0.436 | 0.543 | 0.507 | 0.590 | 0.408 |
| S33 | 0.586 | 0.344 | 0.719 | 0.541 | 0.513 | 0.589 | 0.629 |
| S34 | 0.202 | 0.439 | 0.629 | 0.654 | 0.764 | 0.250 | 0.646 |
| S35 | 0.329 | 0.500 | 0.459 | 0.648 | 0.375 | 0.370 | 0.470 |
| S36 | 0.303 | 0.588 | 0.339 | 0.551 | 0.267 | 0.711 | 0.240 |
| S37 | 0.618 | 0.351 | 0.427 | 0.241 | 0.725 | 0.280 | 0.587 |
| S38 | 0.359 | 0.231 | 0.520 | 0.219 | 0.317 | 0.305 | 0.275 |
| S39 | 0.733 | 0.557 | 0.349 | 0.457 | 0.388 | 0.399 | 0.732 |
| S40 | 0.412 | 0.634 | 0.771 | 0.552 | 0.316 | 0.211 | 0.661 |
| S41 | 0.573 | 0.328 | 0.238 | 0.281 | 0.393 | 0.126 | 0.390 |
| S42 | 0.023 | 0.030 | 0.001 | 0.174 | 0.008 | 0.120 | 0.004 |
| S43 | 0.464 | 0.370 | 0.668 | 0.540 | 0.408 | 0.640 | 0.479 |
| S44 | 0.710 | 0.684 | 0.616 | 0.559 | 0.796 | 0.660 | 0.580 |
| S45 | 0.320 | 0.306 | 0.774 | 0.424 | 0.795 | 0.394 | 0.539 |
| S46 | 0.478 | 0.293 | 0.221 | 0.569 | 0.665 | 0.699 | 0.204 |
| S47 | 0.663 | 0.424 | 0.442 | 0.222 | 0.246 | 0.579 | 0.713 |
| S48 | 0.421 | 0.345 | 0.293 | 0.319 | 0.714 | 0.679 | 0.502 |
| S49 | 0.739 | 0.621 | 0.414 | 0.578 | 0.481 | 0.318 | 0.421 |
| S50 | 0.722 | 0.288 | 0.787 | 0.415 | 0.333 | 0.377 | 0.707 |
| S51 | 0.574 | 0.380 | 0.205 | 0.458 | 0.303 | 0.017 | 0.316 |
| S52 | 0.564 | 0.279 | 0.521 | 0.563 | 0.238 | 0.531 | 0.518 |
| S53 | 0.320 | 0.444 | 0.312 | 0.530 | 0.277 | 0.605 | 0.695 |
| S54 | 0.710 | 0.586 | 0.110 | 0.736 | 0.292 | 0.630 | 0.507 |
| S55 | 0.533 | 0.530 | 0.379 | 0.634 | 0.340 | 0.468 | 0.423 |
| S56 | 0.330 | 0.797 | 0.647 | 0.497 | 0.277 | 0.476 | 0.749 |
| S57 | 0.691 | 0.767 | 0.302 | 0.437 | 0.241 | 0.413 | 0.327 |
| S58 | 0.542 | 0.636 | 0.602 | 0.330 | 0.393 | 0.558 | 0.527 |
| S59 | 0.710 | 0.740 | 0.595 | 0.602 | 0.511 | 0.061 | 0.694 |
| S60 | 0.487 | 0.447 | 0.245 | 0.759 | 0.412 | 0.376 | 0.474 |
| S61 | 0.774 | 0.728 | 0.349 | 0.498 | 0.752 | 0.384 | 0.268 |
| S62 | 0.656 | 0.447 | 0.716 | 0.336 | 0.253 | 0.434 | 0.457 |
| S63 | 0.615 | 0.780 | 0.266 | 0.747 | 0.509 | 0.355 | 0.391 |
| S64 | 0.177 | 0.225 | 0.512 | 0.265 | 0.585 | 0.404 | 0.796 |
| S65 | 0.690 | 0.387 | 0.141 | 0.533 | 0.229 | 0.421 | 0.622 |
| S66 | 0.603 | 0.486 | 0.207 | 0.632 | 0.604 | 0.599 | 0.440 |
| S67 | 0.657 | 0.312 | 0.729 | 0.376 | 0.252 | 0.293 | 0.356 |
| S68 | 0.209 | 0.766 | 0.768 | 0.620 | 0.691 | 0.563 | 0.490 |
| S69 | 0.261 | 0.298 | 0.528 | 0.635 | 0.276 | 0.682 | 0.387 |
| S70 | 0.537 | 0.576 | 0.734 | 0.286 | 0.439 | 0.355 | 0.594 |
| mean | 0.494 | 0.472 | 0.495 | 0.483 | 0.476 | 0.483 | 0.488 |
| SD | 0.196 | 0.180 | 0.198 | 0.180 | 0.193 | 0.198 | 0.177 |

The correlation and mean error matrix table between the brain regions and the EEG frequency ranges are shown in Tables 10 and 11. Low beta, mid beta, and SMR power from the pupillary response indicated moderate correlation and had little difference compared to the EEG power band in the FP1 and FP2 regions (r>0.4, ME<1.5).

Beta power from the pupillary response indicated a moderate correlation and had little difference compared to the EEG power band in the F3, F4, and Fz brain regions (r>0.4, ME<1.5). The high beta power from the pupillary response indicated moderate correlation and had little difference compared to the EEG power band in the F7 and F8 brain regions (r>0.4, ME<1.5).

The mu power from the pupillary response indicated moderate correlation and had little difference compared to the EEG power band in the C3, C4, and Cz brain regions (r>0.4, ME<1.5).

Gamma power from the pupillary response indicated moderate correlation and had little difference compared to the EEG power band in the P3 and P4 brain regions (r>0.4, ME<1.5).

Other brain regions and frequency ranges indicated a low correlation and a large difference (r<0.4, ME>1.5). Low beta, mid beta, SMR, beta, high beta, mu, and gamma were the higher correlations and had very little difference (r>0.6, ME<0.5) with FP1, FP1, FP1, F3, F8, C4, and P4, respectively.

Table 11 shows average of correlation matrix between brain regions and EEG frequency ranges in NMC (dark grey shade r>0.6, light grey shade r>0.4).

Table 12 shows average of mean error matrix between brain regions and EEG frequency ranges in NMC (dark grey shade ME>0.5, light grey shade ME>1.5).

The real-time system for detecting human vital signs was developed using the pupil image from an infrared webcam. This system consisted of an infrared webcam, near IR (Infra-Red light) illuminator (IR lamp) and personal computer for analysis.

The infrared webcam was divided into two types, the fixed type, which is a common USB webcam, and the portable type, which are represented by wearable devices. The webcam was a HD Pro C920 from Logitech Inc. converted into an infrared webcam to detect the pupil area.

The IR filter inside the webcam was removed and an IR passing filter used for cutting visible light from Kodac Inc., was inserted into the webcam to allow passage of IR wavelength longer than 750 nm, as shown in FIG. 12. The 12-mm lens inside the webcam was replaced with a 3.6-mm lens to allow for focusing on the image when measuring the distance from 0.5 m to 1.5 m.

FIG. 12 shows an infrared webcam system for taking pupil images.

The conventional 12 mm lens of the USB webcam shown in FIG. 12 was replaced with a 3.6 mm lens so that the subject could be focused when a distance of 0.5 m to 1.5 m was photographed.

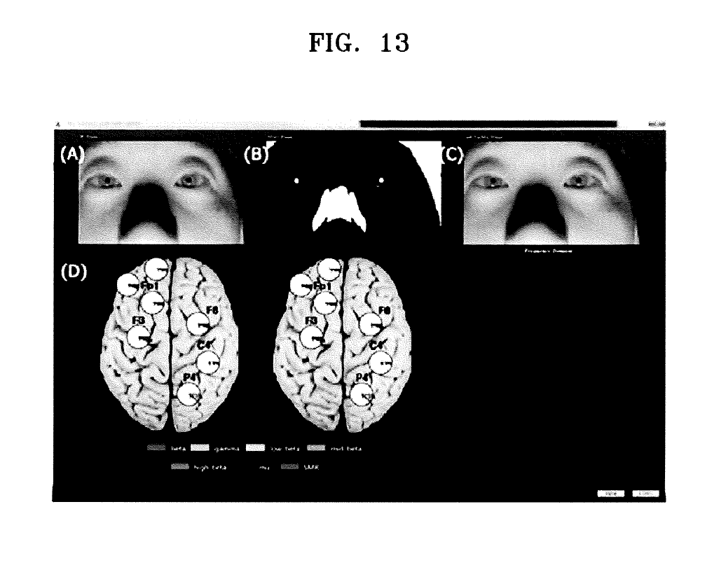

FIG. 13 shows an interface screen of a real-time system for detecting and analyzing a biological signal from an infrared webcam and a sensor.

In FIG. 13, (A) is Infrared pupil image (input image), (B) is binarized pupil image, (C) Detecting the pupil area, and (D) is Output of EEG spectral parameters (low beta power in FP1, mid beta power in FP1, SMR power in FP1, beta power in F3, high beta power in F8, mu power in C4, and gamma power in P4).

As described in the above, the present invention develops and provides an advanced method for non-contact measurements of human vital signs from moving images of the pupil. Thereby, the measurement of parameters in cardiac time domain can be performed by using a low-cost infrared webcam system that monitored pupillary rhythm. The EEG spectral indexes presents the low beta power, mid beta power, and SMR power in FP1 region, beta power in F3 region, high beta power in F8 region, mu power in C4 region, and gamma power in P4 region.

This result was verified for both the conditions of noise (MNC and NMC) and various physiological states (variation of arousal and valence level by emotional stimuli of sound) for seventy subjects.

The research for this invention examined the variation in human physiological conditions caused by the stimuli of arousal, relaxation, positive, negative, and neutral moods during verification experiments. The method based on pupillary response according to the present invention is an advanced technique for vital sign monitoring that can measure vital signs in either static or dynamic situations.

The proposed method according to the present invention is capable of measuring parameters in cardiac time domain with a simple, low-cost, non-invasive, and non-contact measurement system. The present invention may be applied to various industries such as U-health care, emotional ICT, human factors, HCI, and security that require VSM technology. Additionally, it should have a significant ripple effect in terms implementation of non-contact measurements.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. Method of inferring EEG spectrum based on pupillary variation, the method comprising:
obtaining moving images of at least one pupil from a subject;
extracting data of pupillary variation from the moving images;
extracting band data for a plurality of frequency bands to be used as brain frequency information, based on frequency analysis of the signal of pupillary variation; and
calculating outputs of the band data to be used as parameters of a brain-frequency domain.

2. The method of claim 1, wherein the data of pupillary variation comprises a signal indicating pupil size variation of the subject.

3. The method of claim 2, wherein the frequency analysis is performed in a range of 0.01 Hz-0.50 Hz.

4. The method of claim 1, wherein the frequency analysis is performed in a range of 0.01 Hz-0.50 Hz.

5. The method of claim 1, further comprising resampling of the data of pupillary variation at a predetermined sampling frequency, before extracting the band data based on the frequency analysis.

6. The method of claim 5, wherein the plurality of frequency bands include at least one of: a delta range of 0.01 Hz˜0.04 Hz, a theta range of 0.04 Hz˜0.08 Hz, an alpha range of 0.08 Hz˜0.13 Hz, a beta range of 0.13 Hz˜0.30 Hz, a gamma range of 0.30 Hz˜0.50 Hz, a slow alpha range of 0.08 Hz˜0.11 Hz, a fast alpha range of 0.11 Hz˜0.13 Hz, a low beta range of 0.12 Hz˜0.15 Hz, a mid beta range of 0.15 Hz˜0.20 Hz, a high beta range of 0.20 Hz˜0.30 Hz, a mu range of 0.09 Hz˜0.11 Hz, a SensoriMotor Rhythm (SMR) wave range of 0.125 Hz˜0.155 Hz, and a total band range of 0.01 Hz˜0.50 Hz.

7. The method of claim 1, wherein the plurality of frequency bands include at least one of: a delta range of 0.01 Hz˜0.04 Hz, a theta range of 0.04 Hz˜0.08 Hz, an alpha range of 0.08 Hz˜0.13 Hz, a beta range of 0.13 Hz˜0.30 Hz, a gamma range of 0.30 Hz˜0.50 Hz, a slow alpha range of 0.08 Hz˜0.11 Hz, a fast alpha range of 0.11 Hz˜0.13 Hz, a low beta range of 0.12 Hz˜0.15 Hz, a mid beta range of 0.15 Hz˜0.20 Hz, a high beta range of 0.20 Hz˜0.30 Hz, a mu range of 0.09 Hz˜0.11 Hz, a SMR wave range of 0.125 Hz˜0.155 Hz, and a total band range of 0.01 Hz˜0.50 Hz.

8. The method of claim 7, wherein each of the outputs is obtained from a ratio of respective band power to total band power of the total band range.

9. The method of claim 1, wherein each of the outputs is obtained from a ratio of respective band power to total band power of a total band range in which the plurality of frequency bands are included.

10. A system adopting the method of claim 1, comprising:
video equipment configured to capture the moving images of the subject; and
a computer architecture based analyzing system, including analysis tools, configured to process and analyze the moving images in the plurality of frequency bands.

11. The system of claim 10, wherein the analyzing system is configured to perform frequency analysis in a range of 0.01 Hz-0.50 Hz.

12. The system of claim 11, wherein the plurality of frequency bands include at least one of: a delta range of 0.01 Hz˜0.04 Hz, a theta range of 0.04 Hz˜0.08 Hz, an alpha range of 0.08 Hz˜0.13 Hz, a beta range of 0.13 Hz˜0.30 Hz, a gamma range of 0.30 Hz˜0.50 Hz, a slow alpha range of 0.08 Hz˜0.11 Hz, a fast alpha range of 0.11 Hz˜0.13 Hz, a low beta range of 0.12 Hz˜0.15 Hz, a mid beta range of 0.15 Hz˜0.20 Hz, a high beta range of 0.20 Hz˜0.30 Hz, a mu range of 0.09 Hz˜0.11 Hz, a SMR wave range of 0.125 Hz˜0.155 Hz, and a total band range of 0.01 Hz˜0.50 Hz.

13. The system of claim 12, wherein the analyzing system is further configured to calculate each of the outputs from a ratio of respective band power to total band power of the total band range.
